(19) United States
(12) Patent Application Publication
(10) Pub. No.: US 2005/0239217 A1
(43) Pub. Date: Oct. 27, 2005

(54) Fluorogenic homogeneous binding assay methods and compositions

(75) Inventors: Ronald J. Graham, San Ramon, CA (US); Michael M.A. Sekar, Palo Alto, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 10/997,694

(22) Filed: Nov. 24, 2004

Related U.S. Application Data
(60) Provisional application No. 60/525,252, filed on Nov. 26, 2003.

(51) Int. Cl. G01N 33/544

(52) U.S. Cl. 436/528

(57) ABSTRACT

Disclosed are binding substrate compositions, methods and kits useful for, among other things, detecting and/or characterizing binding interactions between molecules of interest.

### 1. CROSS-REFERENCES TO RELATED APPLICATIONS

This application claims benefit under 35 U.S.C. § 119(e) to application Ser. No. 60/525,252, entitled “Fluorogenic Homogeneous Binding Assay Methods and Compositions,” filed Nov. 26, 2003, the disclosure of which is incorporated herein by reference in its entirety.

### 2. FIELD OF THE INVENTION

The present disclosure relates to compositions and methods for detecting and/or characterizing binding interactions.

### 3. INTRODUCTION

Binding interactions between molecules such as ligands and receptors mediate numerous biological processes. For example, many disease pathways are effected by the binding of a ligand to a receptor, which can either “turn on” or “turn off” a cascade of events that leads to manifestation of the disease. The ability to identify ligands for newly identified receptors, or to identify compounds that inhibit binding interactions between ligands and receptors is extremely desirable. For example, compounds that act as competitive inhibitors of ligand-receptor interactions, or compounds that can disrupt or inhibit protein-protein interactions might have clinical or other significances. The ability to detect, to identify, characterize, and screen for binding interactions and/or compounds capable of inhibiting or disrupting binding interactions is therefore desirable.

### 4. SUMMARY

Provided herein are compositions and methods useful for, among other things, detecting and/or characterizing binding interactions between molecules. The methods generally involve contacting a sample with a composition comprising at least one binding substrate that comprises: (a) a binding moiety; (b) a hydrophobic moiety capable of integrating the binding substrate into a micelle; and (c) a fluorescent moiety under conditions effective to permit binding between the binding moiety and a binding partner therefor. When the binding moiety is integrated into a micelle, the fluorescence of its fluorescent moiety is quenched. The sample can include one or a plurality of compounds whose ability to bind the binding moiety, or whose binding characteristics with the binding moiety, are desired to be determined. Following contact, the sample is assessed for an increase or decrease in fluorescence. Whether the sample is assessed for an increase or decrease in fluorescence will depend, in part, upon whether the methods are carried out in the presence or absence of a known binding partner for the binding moiety (e.g, whether the composition further includes a known binding partner for the binding moiety, or whether the binding substrate is included in the composition in complex with a binding partner therefor). For example, if the methods are carried out in the absence of a known binding partner for the binding moiety, the sample can be assessed for an increase in fluorescence, where an increase in fluorescence correlates with the presence of a binding partner for the binding moiety in the sample. If the methods are carried out in the presence of a known binding partner for the binding moiety, the sample can be assessed for a decrease in fluorescence, where a decrease in fluorescence correlates with the presence of a competitive inhibitor of the binding moiety-binding partner complex in the sample. Thus, depending upon how the methods are carried out, they can be used to, among other things: (i) detect, screen for, identify, quantitate and/or characterize compounds that bind the binding moiety; and (ii) detect, screen for, identify, quantitate and/or characterize compounds that modulate the binding activity between the binding moiety and a binding partner therefor, such as, for example, compounds that modulate the binding interaction between the binding moiety and a binding partner therefore.

The binding moiety on the binding substrate can include any molecule of interest (or portion or fragment thereof) for which a binding partner is known or desired, or for which a modulator of binding activity, such as an inhibitor, is desired. For example, the binding moiety may include a small organic molecule, a drug, a hapten, a vitamin, a peptide, a toxin, a hormone, an enzyme, a substrate, a transition state analog, a protein, a protein receptor, an antigen, a ligand, a cytokine, a growth factor, an antibody, a mono- or polysaccharide, or a nucleic acid, including, for example, an oligo- or polynucleotide (e.g., an mRNA, a cDNA or a gene) or nucleic acid analog analog and/or mimic (e.g., a PNA or LNA). In some embodiments, the binding moiety includes one member of a pair of specific binding molecules, such as, for example, one member of a receptor-ligand pair. In some embodiments, the binding moiety includes a molecule whose ability to bind another molecule is sought to be determined. As a specific example, the binding moiety may comprise a receptor (or a binding fragment or domain thereof) whose natural ligand is unknown, or whose natural ligand is known and for which an inhibitor (e.g., a competitive inhibitor, a non-competitive inhibitor, a suicide inhibitor, etc.) is desired. As another specific example, the binding moiety may comprise a small organic molecule, such as a drug lead or candidate, whose ability to bind a protein, receptor or other molecule of interest is sought to be determined.

The binding moiety may be hydrophobic in character, hydrophilic in character, neutral in character or may include one or more regions or portions having hydrophobic, hydrophilic and/or neutral character. In some embodiments, the binding moiety has net hydrophilic character.

The hydrophobic moiety of the binding substrate is capable of integrating the substrate into a micelle. In some embodiments, the hydrophobic moiety comprises a substituted or unsubstituted hydrocarbon comprising from 6 to 30 saturated carbon atoms. In some embodiments, the hydrophobic moiety comprises a phospholipid, such as, for example, a glycerophospholipid or a sphingolipid. Other embodiments are discussed further below. In some embodiments, the hydrophobic moiety is chosen to facilitate an increase in fluorescence of the fluorescent moiety upon binding between the binding moiety and another molecule, such that the amplitude of the increase in fluorescence upon binding is greater than would be obtained with the same binding substrate structure lacking the hydrophobic moiety.

The fluorescent moiety may be any fluorescent entity that is operative in accordance with the compositions and methods described herein. In some embodiments, the fluorescent moiety comprises a fluorescein. In some embodiments, the fluorescent moiety comprises a sulfofluorescein. In some embodiments, the fluorescent moiety comprises a rhodamine. Other fluorescent moieties may also be used.

The binding moiety, hydrophobic moiety and fluorescent moiety are connected in any way that permits them to perform their respective functions. In some embodiments, the hydrophobic moiety and the fluorescent moiety are linked to each other through the binding moiety. In other embodiments, the hydrophobic moiety and the binding moiety are linked to each other through the fluorescent moiety. In still other embodiments, a trivalent linker links the hydrophobic moiety, the fluorescent moiety and the binding moiety.

The composition may include a single binding substrate, or it may include a plurality of different binding substrates. When the composition includes a plurality of different binding substrates, the substrates may differ from one another with respect to any one or more of their binding moieties, hydrophobic moieties and/or fluorescent moieties. As a specific example, the composition can include two binding substrates that differ from one another with respect to at least their fluorescent moieties. In some embodiments, the different fluorescent moieties can be selected such that their fluorescence spectra are resolvable from another. For example, the fluorescent moiety on a first binding substrate can be selected to fluoresce in the green region of the spectrum and the fluorescent moiety on a second binding substrate selected to fluoresce in the red region of the spectrum. In some embodiments, the binding substrates can also differ from one another with respect to the identities of their binding moieties, permitting the ability to carry out the methods in a “multiplexed” fashion, where binding moieties capable of binding different molecules are correlated with a particular fluorescence signal. When binding substrates having such spectrally resolvable fluorescent moieties are used, the fluorescent moieties can be selected to have different absorbance or excitation spectra or maxima, or all or a subset may be selected to have similar absorbance or excitation spectra or maxima such that they can be simultaneously excited with a single excitation source.

When a plurality of different binding substrates are used, although not required for operation, the fluorescent moieties on one or more of the substrates can be selected such that they quench the fluorescence of the fluorescent moieties on one or more of the other substrates when the moieties are in close proximity to one another such as, for example, by orbital mixing (i.e., forming a ground state dark complex), collisional quenching, fluorescence resonance energy transfer (FRET) or by another mechanism (or combination of mechanisms). As a specific example, the fluorescent moiety of a first binding substrate can be selected that has an absorbance spectrum that sufficiently overlaps the emissions spectrum of the fluorescent moiety of a second binding substrate such that the first fluorescent moiety substantially quenches the fluorescence of the second fluorescent moiety when the two are in close proximity to one another, such as when both binding substrates are integrated into the same micelle. As another specific example, the fluorescent moieties of two (or more) different binding substrates may be selected such that they quench the fluorescence of each other when in close proximity thereto.

Although not required for operation, the composition may optionally include one or more amphipathic quenching molecules capable of quenching the fluorescence of a fluorescent moiety on a binding substrate when the binding substrate and the quenching molecule are in close proximity to one another, such as when the binding substrate and quenching molecule are integrated into the same micelle. Such quenching molecules generally comprise a hydrophobic moiety capable of integrating the quenching molecule into a micelle and a quenching moiety. Specific embodiments of the hydrophobic moiety can include any of the hydrophobic moieties discussed above in connection with the binding substrates.

The quenching moiety can be any moiety capable of quenching the fluorescence of a fluorescent moiety on a binding substrate. In some embodiments, the quenching moiety can itself be a fluorescent moiety that is capable of quenching the fluorescence of the fluorescent moiety on a binding substrate when placed in close proximity thereto, such as, for example, by orbital mixing, collisional quenching, fluorescence resonance energy transfer (FRET) or by another mechanism (or combination of mechanisms). As a specific example, the quenching moiety can be a fluorescent moiety having an absorbance spectrum that sufficiently overlaps the emissions spectrum of the fluorescent moiety on a binding substrate such that the quenching moiety substantially quenches the fluorescence of the binding substrate fluorescent moiety when the quenching moiety and fluorescent moiety of the binding substrate are in close proximity to one another, such as when the quenching molecule and binding substrate are integrated into the same micelle. In other embodiments, the quenching moiety is non-fluorescent. The quenching molecule can optionally include a binding moiety, which can be the same as or different from the binding moiety of the binding substrate.

Also provided are binding substrates and compositions and kits comprising them, as discussed further herein.

The methods and compositions may be used in a variety of contexts, including, for example, to detect, characterize, screen for, quantify and/or identify binding partners for molecules of interest or to detect, characterize, screen for, quantify and/or identify inhibitors of molecules of interest (e.g., competitive inhibitors, non-competitive inhibitors, suicide inhibitors, etc.), as discussed further herein.

These and other features of the compositions and methods will become more apparent from the Description.

### 5. BRIEF DESCRIPTION OF THE DRAWINGS

The skilled artisan will understand that the drawings, described below, are for illustration purposes only. The drawings are not intended to limit the scope of the present teachings in any way.

FIGS. 1A-1F illustrate exemplary embodiments of binding substrates in which the binding moiety, fluorescent moiety and hydrophobic moiety are linked together via a trivalent lysine linker;

FIG. 1G illustrates exemplary embodiments of trivalent linker synthons suitable for forming trivalent linkers;

FIG. 2A illustrates an exemplary method for synthesizing the binding substrate of FIG. 1A;

FIGS. 3A and 3B illustrate exemplary embodiments of binding substrates according to FIGS. 1A and 1B, respectively, in which the hydrophobic moiety (—NH—R¹) is provided by a glycerophospholipid (phosphatidylethanolamine);

FIGS. 4A-4D illustrate specific embodiments of binding substrates according to FIGS. 1A-1C and 1F, respectively;

FIGS. 5A-5D illustrate exemplary methods of synthesizing the binding substrates of FIGS. 4A-4D, respectively;

FIG. 6 provides a cartoon illustrating the principles of a binding assay carried out with an exemplary binding substrate;

FIGS. 7A-7D provide electrospray ionization (ESI) mass spectra for Compounds 104, 106A, 106 and 107, respectively (illustrated in FIG. 5C);

FIG. 8 provides an ESI mass spectrum of Compound 111 (illustrated in FIG. 5B);

FIG. 9 provides an ESI mass spectrum of Compound 103 (illustrated in FIG. 5A);

FIG. 10 provides a graph illustrating the increase in fluorescence observed with the exemplary binding substrate of FIG. 4C (Compound 107) in the presence of anti-thyroxine monoclonal antibodies;

FIG. 11 provides a graph illustrating the increase in fluorescence observed with the exemplary binding substrate of FIG. 4B (Compound 111) in the presence of recombinant enzymatically inactive COX-2 apoenzyme; and

FIG. 12 provides a graph illustrating the increase in fluorescence observed with the exemplary binding substrate of FIG. 4A (Compound 103) in the presence of streptavidin.

### 6. DESCRIPTION OF VARIOUS EMBODIMENTS

### 5.1 Definitions

Unless stated otherwise, the following terms and phrases used herein are intended to have the following meanings:

“Detect” and “detection” have their standard meaning, and are intended to encompass detection, measurement, and/or characterization of a selected molecule or molecular activity. As a specific example, binding activity or interactions may be “detected” in the course of detecting the presence of, screening for, quantifying, identifying or characterizing binding partners or inhibitors of a binding substrate.

“Fatty Acid” has its standard meaning and is intended to refer to a long-chain hydrocarbon carboxylic acid in which the hydrocarbon chain is saturated, mono-unsaturated or polyunsaturated. The hydrocarbon chain may be linear, branched or cyclic, or may include a combination of these features, and may be unsubstituted or substituted. Fatty acids typically have the structural formula R—C(O)OH, where R is a substituted or unsubstituted, saturated, mono-unsaturated or polyunsaturated hydrocarbon comprising from 6 to 30 carbon atoms which has a structure that is linear, branched, cyclic or a combination thereof.

“Micelle” has its standard meaning and is intended to refer to an aggregate formed by amphipathic molecules in water or an aqueous solvent such that their polar ends or portions are in contact with the water or aqueous solvent and their nonpolar ends or portions are in the interior of the aggregate. A micelle can take any shape or form, including but not limited to, a non-lamellar “detergent-like” aggregate that does not enclose a portion of the water or aqueous solvent, or a unilamellar or multilamellar “vesicle-like” aggregate that encloses a portion of the water or aqueous solvent, such as, for example, a liposome.

“Phospholipid” has its standard meaning and is intended to include compounds that comprise two fatty acid components, a backbone component, a phosphate component and an organic component. Specific examples of phospholipids include glycerophospholipids and sphingolipids. Specifically included within the definition of “phospholipid” are glycerophospholipids having the following structure: 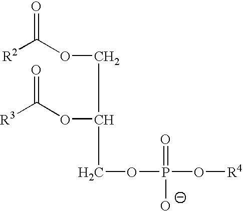
wherein:
R² is a saturated, mono-unsaturated or polyunsaturated hydrocarbon comprising form 6 to 30 carbon atoms;
R³ is a saturated, mono-unsaturated or polyunsaturated hydrocarbon comprising form 6 to 30 carbon atoms; and
R⁴ is —CH₂CH₂—N⁺(CH₃)₃ (cholinyl), —CH₂CH₂NH₂(ethanolamin-2-yl), inositotyl —CH₂CH(NH₃⁺)C(O)OH (serinyl) or —CH₂CH(NH₂)—CH(OH)—CH═CH—(CH₂)₁₂CH₃.

“Quench” has its standard meaning and is intended to refer to a measurable reduction in the fluorescence intensity of a fluorescent group or moiety as measured at a specified wavelength, regardless of the mechanism by which the reduction is achieved. As specific examples, the quenching may be due to orbital overlap, molecular collision, energy transfer such as FRET, a change in the fluorescence spectrum (color) of the fluorescent group or moiety or any other mechanism (or combination of mechanisms). The amount of the reduction is not critical and may vary over a broad range. The only requirement is that the reduction be measurable by the detection system being used. Thus, a fluorescence signal is “quenched” if its intensity at a specified wavelength is reduced by any measurable amount. A fluorescence signal is “substantially quenched” if it is reduced by at least 50%, 60%, 70%, 75%, 80%, 85%, 90%, 95%, 96%, 97%, 98%, 99% or even 100% as compared to a reference control sample or a reference value.

Polypeptide sequences are provided with an orientation (left to right) of the N terminus to C terminus, with amino acid residues represented by the standard 3-letter or 1-letter codes (e.g., Stryer, L., Biochemistry, 2^{nd} Ed., W.H. Freeman and Co., San Francisco, Calif., page 16 (1981)).

### 5.2 Binding Substrate Compositions

### 5.2.1 Binding Substrates Per Se

In one aspect, binding substrates are provided that can be designed to detect binding interactions between any of a large variety of different types of molecules. The substrates include a binding moiety, a hydrophobic moiety and a fluorescent moiety. The hydrophobic moiety is capable of integrating the binding substrate into a micelle. When integrated in the micelle, the fluorescence of the fluorescent moiety is quenched and the binding moiety is positioned such that it is available to, or capable of, binding another molecule present in a sample. Upon binding between the binding moiety and another molecule, such as a known binding partner for the binding moiety or a candidate molecule of interest present in a sample that can bind the binding moiety, the fluorescence of the fluorescent moiety become unquenched, and increases in intensity. As a consequence of this property, the binding substrates and/or micelles comprising them can be used in a variety of assays, such as for example, assays to assess and/or characterize binding interactions between the binding moiety and other molecules. In some embodiments, the binding substrates and methods may be used in a continuous monitoring phase, in real time, to allow the user to rapidly determine whether binding interactions, binding molecules and/or modulators of binding interactions such as, for example, competitive inhibitors are present in a sample.

The binding moiety of the binding substrate can include virtually any molecule of interest, the identity of which will depend, in large part, on the desired application for the binding substrate. For example, if the binding substrate will be used to screen samples for the presence of a receptor of interest, the binding moiety can include a known ligand for the receptor. If the binding substrate will be used to screen for and/or identify a binding partner for a molecule of interest, the binding moiety may include either the molecule of interest (or a binding fragment or portion thereof) or a putative (candidate) binding partner. If the binding substrate will be used to screen for, identify and/or characterize an inhibitor of a pair of binding molecules, such as a receptor-ligand pair, the binding moiety can include either the ligand or the receptor (or a binding domain or fragment thereof). Thus, depending upon the application, the binding moiety can include, by way of example and not limitation, a small organic molecule, a drug, a hapten, a vitamin, a peptide, a protein, a toxin, a hormone, an enzyme, an enzyme substrate, a transition state analog, a receptor, a ligand, an antigen, a cytokine, a growth factor, an antibody, a mono- or polysaccharide, a nucleic acid (e.g., an oligo- or polynucleotide, an mRNA, a cDNA, a gene, etc.), a nucleic acid analog or mimic (e.g., a PNA or LNA) or binding portions, domains or fragments thereof.

In embodiments in which the binding moiety comprises one member of an enzyme-substrate pair, chemical modification of the binding partners may be permitted, provided that the desired binding event that will be detected is not significantly compromised. In some embodiments in which the binding moiety comprises one member of an enzyme-substrate pair, it may be desirable to adjust the binding assay conditions so that the binding moiety can bind, but not chemically modify or be chemically modified by, its binding partner. Numerous enzymes are known that require cofactors for enzymatic activity. As specific non-limiting examples, many endonuclease enzymes will bind, but not cleave, nucleic acid sequences in the absence of ions such as Mg²⁺; protein kinases will bind, but not phosphorylate, peptides and proteins in the absence of ATP; COX-2 apoenzyme will bind, but not act upon, arachidonic acid in the absence of a heme group. In some embodiments, it may be desirable to carry out a binding assay in the absence of a cofactor(s) required for enzymatic activity (or at a concentration of cofactor(s) below that required for activity.

Particularly important pairs of binding molecules include ligand-receptor pairs that are involved in signaling cascades and/or disease pathways. In some embodiments, the binding moiety includes one member of a pair of such ligand-receptor pairs. A wide variety of such pairs of molecules are known and include, by way of example and not limitation, folate/folate receptor, thyroxine/thyroxine receptor, methotrexate/DHFR, dexamethasone/glucocorticoid receptor, estradiol/estrogen receptor, phallodin/F-actin, geldanamucin/heatshock protein 90, progesterone/progesterone receptor, testosterone/testosterone receptor, D-myo-inosistol 1,4,5-triphosphate (IP₃)/IP₃ receptor, forskolin/multidrug resistance protein, verapamil/multidrug resistance protein, pirenzepenine/muscarinic acetylcholine receptor, muscimol/γ-aminobutyric acid A receptor, naloxone/μ-opioid receptor, prazosin/α₁-adrenergic receptor, and ouabain/Na⁺/K⁺ ATPase. The binding moiety may include either the ligand or the receptor (or a binding domain or portion thereof), depending upon the particular application. In some embodiments, the binding moiety includes the ligand of a ligand-receptor pair.

In addition to the binding moiety, the binding substrate also comprises a hydrophobic moiety capable of anchoring or integrating the binding substrate into a micelle. The exact number, lengths, sizes and/or composition of the hydrophobic moiety(ies) can be selectively varied. In some embodiments, the hydrophobic moiety comprises a substituted or unsubstituted hydrocarbon of sufficient hydrophobic character (e.g., length and/or size) to cause the binding substrate to become integrated or incorporated into a micelle when the binding substrate is dispersed in an aqueous solvent at a concentration above a micelle-forming threshold, such as at or above its critical micelle concentration (CMC). In some embodiments, the hydrophobic moiety comprises a substituted or unsubstituted hydrocarbon comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The substituted or unsubstituted hydrocarbon may be linear, branched, cyclic, or any combination thereof. In some embodiments, the hydrocarbon is unsubstituted. In some embodiments, the hydrocarbon is substituted with one or more halogens, such as one or more F, Cl or Br groups. Exemplary linear unsubstituted hydrocarbon groups include C6, C7, C8, C9, C10, C11, C12, C13, C14, C15, C16, C17, C18, C19, C20, C22, C24, and C26 alkyl chains. Exemplary linear substituted hydrocarbon groups include C6, C7, C8, C9, C10, C11, C12, C13, C14, C15, C16, C17, C18, C19, C20, C22, C24 and C26 fluorinated or perfluorinated alkyl chains.

In some embodiments, the hydrophobic moiety is fully saturated. In other embodiments, the hydrophobic moiety comprises one or more carbon-carbon double bonds which may be, independently of one another, in the cis or trans configuration, and/or one or more carbon-carbon triple bonds. In some cases, the hydrophobic moiety may include one or more cycloalkyl groups (e.g., cyclopropyl, cyclobutyl, cyclopentyl, cyclohexyl groups), or one or more aryl rings or arylalkyl groups, such as one or two phenyl or benzyl groups.

In some embodiments, the hydrophobic moiety is a nonaromatic group that does not have a cyclic aromatic pi electron system. In some embodiments, if the hydrophobic moiety contains one or more unsaturated carbon-carbon bonds, those carbon-carbon bonds are not conjugated. In some embodiments, the structure of the hydrophobic moiety is incapable of interacting with the fluorescent moiety, by a FRET or stacking interaction, to quench fluorescence of the fluorescent moiety. Also encompassed herein are embodiments that involve a combination of any two or more of the foregoing embodiments. Optimization testing can be done by making several binding substrates having different hydrophobic moieties.

For embodiments in which the hydrophobic moiety is linked to the fluorescent moiety, it will be understood that the hydrophobic moiety is distinct from the fluorescent moiety because the hydrophobic moiety does not include any of the atoms in the fluorescent moiety that are part of the aromatic or conjugated pi-electron system that produces the fluorescent signal. Thus, if a hydrophobic moiety is connected to the 4 position of a xanthene ring (4′-position of a rhodamine or fluorescein), the hydrophobic moiety does not include any of the aromatic ring atoms of the xanthene ring.

It is to be understood that the hydrophobic moiety is distinct from the binding moiety. In some instances, a binding moiety may have sufficient hydrophobic character that when it is linked to a hydrophilic molecule, such as a hydrophilic fluorescent dye, the resultant conjugate is amphiphilic and can form a micelle in aqueous solution. These types of molecules, in which the binding moiety plays a dual role, are not contemplated as binding molecules herein. The binding molecules described herein include at least three distinct “domains” or “regions”—a binding moiety, a fluorescent moiety and a hydrophobic moiety.

Moreover, the hydrophobic moiety is capable of integrating the binding substrate into a micelle. Thus, the hydrophobic moiety is distinct from a hydrocarbon linkage linking a fluorescent moiety to a binding moiety. To function properly, the hydrophobic moiety should include an end that is not attached to another moiety of the binding substrate. Molecules which include a binding domain linked to a fluorescent dye via a hydrocarbon linkage that are not binding substrates as defined herein are described in Farinas & Verkman, 1999, J. Biol. Chem. 274(12):7603-7606; Timofeevski et al., 2002, Biochemistry 41:9654-9662; and Adamczyk et al., 2002, Bioorg. Med. Chem. Lett. 12:1283-1285. All of these compounds lack a hydrophobic moiety as defined herein.

As will be described in more detail below, in some embodiments, the binding substrate is an analog or a derivative of a phospholipid, for example, a glycerophospholipid. In such embodiments, the binding substrate typically includes two hydrocarbon moieties linked to the C1 and C2 carbons of a glycerolyl group via ester linkages (or other linkages). In such embodiments, the two hydrocarbon moieties may be the same, or they may differ from another. In some embodiments, each hydrocarbon moiety is selected to correspond to the hydrocarbon chain or “tail” of a naturally occurring fatty acid. In another specific embodiment, the hydrocarbon moieties are selected to correspond to the hydrocarbon chains or tails of a naturally occurring glycerophospholipid. Non-limiting examples of useful hydrocarbon chains or tails of commonly occurring fatty acids are provided in Table 1, below:

TABLE 1

| Length:Number | |
|---|---|
| of Unsaturations | Common Name |
| 14:0 | myristic acid |
| 16:0 | palmitic acid |
| 18:0 | stearic acid |
| 18:1 cisΔ⁹ | oleic acid |
| 18:2 cisΔ^{9,12} | linoleic acid |
| 18:3 cisΔ^{9,12,15} | linonenic acid |
| 20:4 cisΔ^{5,8,11,14} | arachidonic acid |
| 20:5 cisΔ^{5,8,11,14,17} | eicosapentaenoic acid (an omega-3 fatty acid) |

While the basis for the change in fluorescence observed in assays utilizing the binding substrates described herein may not be certain, it is contemplated that, in the absence of a binding partner for the binding moiety, the binding substrates are capable of forming micelles in aqueous buffer due their hydrophobic moieties. When integrated into a micelle, the fluorescent moieties on different binding substrates quench each other due to their close proximity and high local concentration. Micelle formation may be evidenced by an increase in light scatter by a shift in the absorbance maximum of the fluorescent moiety and/or by an observed increase in fluorescence upon addition of a surfactant, such as, for example, Triton X-100, at a concentration that disrupts micelle formation. In experiments performed in support of the compositions and methods described herein, addition of Triton X-100 to an aqueous solution of an exemplary binding substrate resulted in an observed increase in fluorescence (see, e.g., Section 6.4, infra). However, it is possible that actual formation of micelles by the binding substrates is not required for operability.

The fluorescent moiety in the binding substrate may be any entity that provides a fluorescent signal that can be used to follow binding interactions. Typically, the fluorescent moiety comprises a fluorescent dye that in turn comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes.

In some embodiments, the fluorescent moiety comprises a xanthene dye. Generally, xanthene dyes are characterized by three main features: (1) a parent xanthene ring; (2) an exocyclic hydroxyl or amine substituent; and (3) an exocyclic oxo or imminium substituent. The exocyclic substituents are typically positioned at the C3 and C6 carbons of the parent xanthene ring, although “extended” xanthenes in which the parent xanthene ring includes a benzo group fused to either or both of the C5/C6 and C3/C4 carbons are also known. In these extended xanthenes, the characteristic exocyclic substituents are positioned at the corresponding positions of the extended xanthene ring. Thus, as used herein, a “xanthene dye” generally comprises one of the following parent rings:

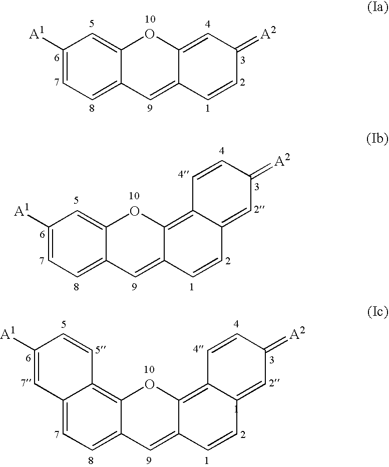

In the parent rings depicted above, A¹ is OH or NH₂ and A² is O or NH₂⁺. When A¹ is OH and A² is O, the parent ring is a fluorescein-type xanthene ring. When A¹ is NH₂ and A² is NH₂⁺, the parent ring is a rhodamine-type xanthene ring. When A¹ is NH₂ and A² is O, the parent ring is a rhodol-type xanthene ring.

One or both of nitrogens of A¹ and A² (when present) and/or one or more of the carbon atoms at positions C1, C2, C2′, C4, C4′, C5, C5′, C7′, C7 and C8 of the par rings can be independently substituted with a wide variety of the same or different substituents. In some embodiments, typical substituents include, but are not limited to, —X, —R^{a}, —OR^{a}, —SR^{a}, —NR^{a}R^{a}, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R^{a}, —C(O)R, —C(O)X, —C(S)R^{a}, —C(S)X, —C(O)OR^{a}, —C(O)O⁻, —C(S)OR^{a}, —C(O)SR^{a}, —C(S)SR^{a}, —C(O)NR^{a}R^{a}, —C(S)NR^{a}R^{a} and —C(NR)NR^{a}R^{a}, where each X is independently a halogen (preferably —F or —Cl) and each R^{a} is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, 5-20 membered heteroaryl, 6-26 membered heteroarylalkyl, 5-20 membered heteroaryl-heteroaryl, carboxyl, acetyl, sulfonyl, sulfinyl, sulfone, phosphate, or phosphonate. Generally, substituents which do not tend to completely quench the fluorescence of the parent ring are preferred, but in some embodiments quenching substituents may be desirable. Substituents that tend to quench fluorescence of parent xanthene rings are electron-withdrawing groups, such as —NO₂, —Br and —I.

The C1 and C2 substituents and/or the C7 and C8 substituents can be taken together to form substituted or unsubstituted buta[1,3]dieno or (C₅-C₂₀) aryleno bridges. For purposes of illustration, exemplary parent xanthene rings including unsubstituted benzo bridges fused to the C1/C2 and C7/C8 carbons are illustrated below:

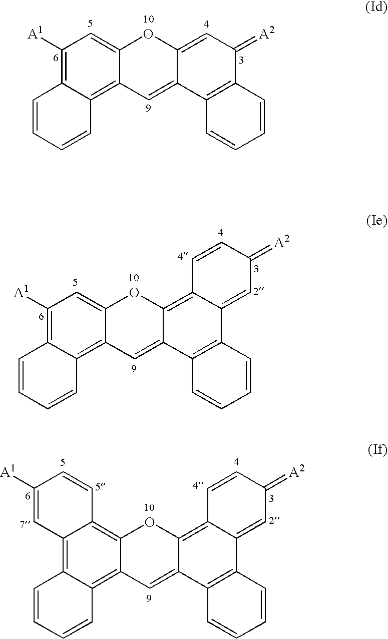

The benzo or aryleno bridges may be substituted with a variety of different substituent group, at one or more positions, such as with the substituent groups previously described above for carbons C1-C8 in structures (Ia)-(Ic), supra. In embodiments including a plurality of substituents, the substituents may all be the same, or some or all of the substituents can differ from one another.

When A¹ is NH₂ and/or A² is NH₂⁺, the nitrogen atoms may be included in one or two bridges involving adjacent carbon atom(s). The bridging groups may be the same or different, and are typically selected from (C₁-C₁₂) alkyldiyl, (C₁-C₁₂) alkyleno, 2-12 membered heteroalkyldiyl and/or 2-12 membered heteroalkyleno bridges. Non-limiting exemplary parent rings that include bridges involving the exocyclic nitrogens are illustrated below:

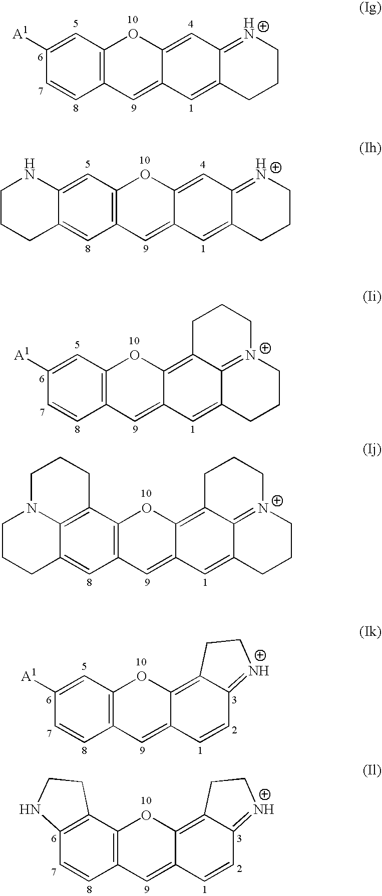

The parent ring may also include a substituent at the C9 position. In some embodiments, the C9 substituent is selected from acetylene, lower (e.g., from 1 to 6 carbon atoms) alkanyl, lower alkenyl, cyano, aryl, phenyl, heteroaryl, electron-rich heteroaryl and substituted forms of any of the preceding groups. In embodiments in which the parent ring includes benzo or aryleno bridges fused to the C1/C2 and C7/C8 positions, such as, for example, rings (Id), (Ie) and (If) illustrated above, the C9 carbon is preferably unsubstituted.

In some embodiments, the C9 substituent is a substituted or unsubstituted phenyl ring such that the xanthene dye comprises one of the following structures:

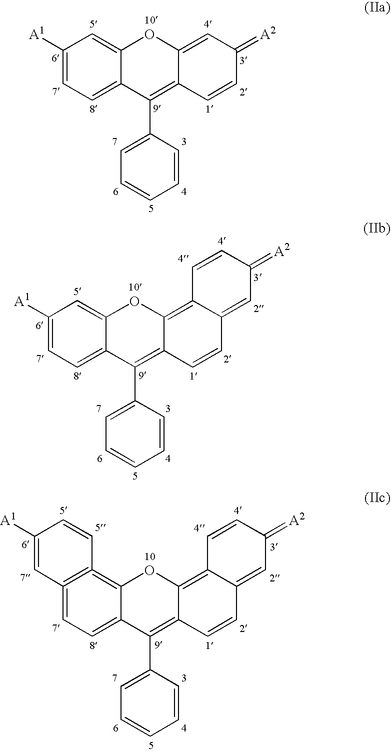

The carbons at positions 3, 4, 5, 6 and 7 may be substituted with a variety of different substituent groups, such as the substituent groups previously described for carbons C1-C8. In some embodiments, the carbon at position C3 is substituted with a carboxyl (—COOH) or sulfuric acid (—SO₃H) group, or an anion thereof. Dyes of formulae (IIa), (IIc) and (IIc) in which A¹ is OH and A² is O are referred to herein as fluorescein dyes; dyes of formulae (IIa), (IIb) and (IIc) in which A¹ is NH₂ and A² is NH₂⁺ are referred to herein as rhodamine dyes; and dyes of formulae (IIa), (IIb) and (IIc) in which A¹ is OH and A² is NH₂⁺ (or in which A¹ is NH₂ and A² is O) are referred to herein as rhodol dyes.

As highlighted by the above structures, when xanthene rings (or extended xanthene rings) are included in fluorescein, rhodamine and rhodol dyes, their carbon atoms are numbered differently. Specifically, their carbon atom numberings include primes. Although the above numbering systems for fluorescein, rhodamine and rhodol dyes are provided for convenience, it is to be understood that other numbering systems may be employed, and that they are not intended to be limiting. It is also to be understood that while one isomeric form of the dyes are illustrated, they may exist in other isomeric forms, including, by way of example and not limitation, other tautomeric forms or geometric forms. As a specific example, carboxy rhodamine and fluorescein dyes may exist in a lactone form.

In one specific embodiment, the fluorescent moiety comprises a fluorescent dye that has net hydrophilic character. In one specific embodiment, the fluorescent moiety comprises a xanthene dye that has net hydrophilic character.

In another specific embodiment, the fluorescent moiety comprises a rhodamine dye. Exemplary suitable rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional suitable rhodamine dyes include, for example, those described in U.S. Pat. Nos. 6,248,884, 6,111,116, 6,080,852, 6,051,719, 6,025,505, 6,017,712, 5,936,087, 5,847,162, 5,840,999, 5,750,409, 5,366,860, 5,231,191, and 5,227,487; PCT Publications WO 97/36960 and WO 99/27020; Lee et al., NUCL. ACIDS RES. 20:2471-2483 (1992), Arden-Jacob, NEUE LANWELLIGE XANTHEN-FARBSTOFFE FÜR FLUORESZENZSONDEN UND FARBSTOFF LASER, Verlag Shaker, Germany (1993), Sauer et al., J. FLUORESCENCE 5:247-261 (1995), Lee et al., NUCL. ACIDS RES. 25:2816-2822 (1997), and Rosenblum et al., NUCL. ACIDS RES. 25:4500-4504 (1997). A particularly preferred subset of rhodamine dyes are 4,7,-dichlororhodamines. In some embodiments, the fluorescent moiety comprises a 4,7-dichloro-orthocarboxyrhodamine dye.

In still another specific embodiment, the fluorescent moiety comprises a fluorescein dye. Exemplary suitable fluorescein include, but are not limited to, fluorescein dyes described in U.S. Pat. Nos. 6,008,379, 5,840,999, 5,750,409, 5,654,442, 5,188,934, 5,066,580, 4,933,471, 4,481,136 and 4,439,356; PCT Publication WO 99/16832, and EPO Publication 050684. A preferred subset of fluorescein dyes are 4,7-dichlorofluoresceins. Other preferred fluorescein dyes include, but are not limited to, 5-carboxyfluorescein (5-FAM) and 6-carboxyfluorescein (6-FAM). In some embodiments, the fluorescein moiety comprises a 4,7-dichloro-orthocarboxyfluorescein dye.

In other embodiments, the fluorescein moiety can include a cyanine, a phthalocyanine, a squaraine, or a bodipy dye, such as those described in the following references and the references cited therein: U.S. Pat. Nos. 6,080,868, 6,005,113, 5,945,526, 5,863,753, 5,863,727, 5,800,996, and 5,436,134; and PCT Publication WO 96/04405.

In still other embodiments, the fluorescent moiety can include a network of dyes that can operate cooperatively with one another such as, for example by FRET or another mechanism, to provide large Stoke's shifts. Such dye networks typically include a fluorescence donor moiety and a fluorescence acceptor moiety, and may include moieties that act as both fluorescence acceptors and donors. The fluorescence donor and acceptor moieties can comprise any of the previously described dyes that can act cooperatively with one another. In some embodiments, the fluorescent moiety comprises a fluorescence donor moiety which comprises a fluorescein dye and a fluorescence acceptor moiety which comprises a fluorescein or rhodamine dye.

The binding moiety, hydrophobic moiety, and fluorescent moiety can be connected in any way that permits them to perform their respective functions. In some embodiments, the hydrophobic moiety and the binding moiety are linked to each other through the fluorescent moiety. In other embodiments, the hydrophobic moiety and the fluorescent moiety are linked to each other through the binding moiety. As a specific example, where the binding moiety includes a polypeptide or polynucleotide segment, the hydrophobic moiety and the fluorescent moiety can be linked to opposite ends of the polypeptide or polynucleotide. In still other embodiments, the hydrophobic moiety, the fluorescent moiety, and the binding moiety are linked by a trivalent linker.

As discussed above, the hydrophobic moiety functions to incorporate the binding substrate into a micelle. Thus, the binding moiety and fluorescent moiety are typically not linked through the hydrophobic moiety—the hydrophobic moiety typically includes a free end or terminus that is not linked to another moiety of the binding substrate.

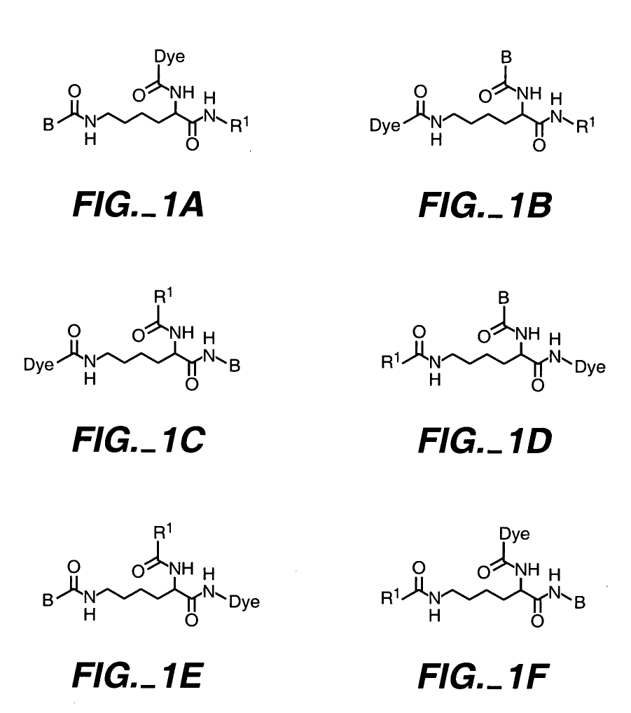

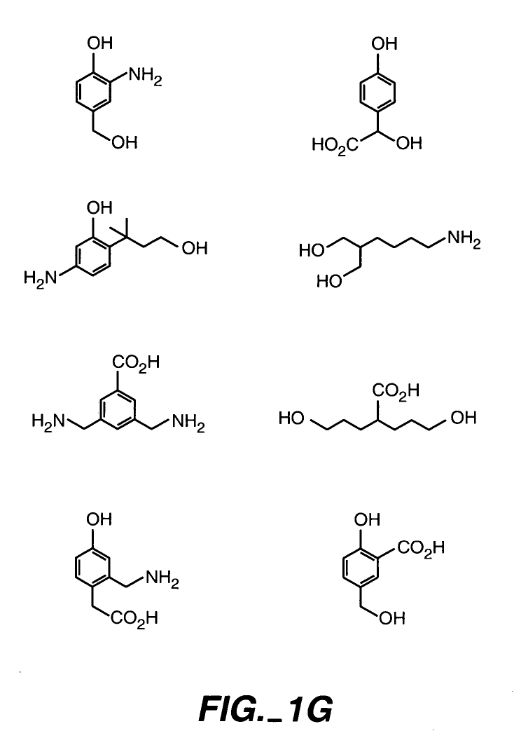

FIG. 1A illustrates an exemplary embodiment of a binding substrate in which the hydrophobic moiety, binding moiety and fluorescent moiety are linked via a linkage provided by a trivalent linker synthon (such a linkage is referred to herein as a “trivalent linker”). In the illustrated substrate, the trivalent linker is provided by the α-amino acid lysine. The binding moiety (B—C(O)—) is linked to the side chain (epsilon) amino group, the fluorescent moiety (Dye-C(O)—) is linked to the alpha amino group and the hydrophobic moiety (R¹—NH—) is linked to the alpha carboxyl. The binding, fluorescent and hydrophobic moieties could be linked to the lysine linker in other arrangements from that illustrated, specific examples of which are illustrated in FIGS. 1B-1F.

As will be appreciated by skilled artisans, in FIGS. 1A-1F, the illustrated lysine is merely an exemplary trivalent linker. Any molecule having three “reactive” groups suitable for attaching other molecules and moieties thereto, or that can be appropriately activated to attach other molecules and moieties thereto, could be used as a trivalent linker synthon to provide a trivalent linker. For example, the “backbone” of the linker synthon to which the reactive (or activatable) linking groups are attached could be a linear, branched or cyclic saturated or unsaturated alkyl, a mono or polycyclic aryl or an arylalkyl. Moreover, while the previous examples are hydrocarbons, the linker backbone need not be limited to carbon and hydrogen atoms. Indeed, the linker backbone can include single, double, triple or aromatic carbon-carbon bonds, carbon-nitrogen bonds, nitrogen-nitrogen bonds, carbon-oxygen bonds, carbon-sulfur bonds and combinations thereof, and therefore can include functionalities such as carbonyls, ethers, thioethers, carboxamides, sulfonamides, ureas, urethanes, hydrazines, etc. Any type of linker backbone that permits the binding substrate to function as described herein may be used.

Pairs of complementary functional groups that are suitable for forming covalent linkages with one another are well-known in the art. The functional groups on a trivalent linker synthon can be any member of such complementary pairs. In some embodiments, each reactive group comprising a trifunctional linker synthon is an electrophilic group or a nucleophilic group that is capable of reacting with a complementary nucleophilic group or electrophilic group to form a covalent linkage stable to biological assay conditions. Specific examples of such complementary pairs electrophilic and nucleophilic groups, as well as the resultant linkages formed therefrom, are provided in Table 2, below:

TABLE 2

| Electrophilic Group | Nucleophilic Group | Resultant Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | esters |
| anhydrides | alcohols/phenols | esters |
| anhydrides | amines/anilines | caroboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphate esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | esters |
| sulfonyl halides | amines/anilines | sulfonamides |
| sulfonyl halides | phenols/alcohols | sulfonate esters |

*Activated esters, as understood in the art, generally have the formula —C(O)Z, where Z is a good leaving group (e.g., oxysuccinimidyl, oxysulfosuccinimidyl, 1-oxybenzotriazolyl, etc.).
**Acyl azides can rearrange to isocyanates

The reactive groups on a trivalent linker synthon may all be the same, or some or all of them may be different. In some embodiments, reactive groups are selected that have different chemical reactivities to facilitate the selective attachment of the binding, fluorescent and hydrophobic moieties, to the linker synthon.

In some embodiments, the trifunctional linker synthon is an amino acid, which may be an alpha amino acid, a beta amino acid, a gamma amino acid or other type of amino acid, that includes a side chain having a suitable reactive functional group. Specific non-limiting examples of suitable amino acids include, but are not limited to, lysine, glutamate, cysteine, serine, homoserine and 1,3-diaminobutyric acid. These amino acids may be in either the D- or L-configuration, or may constitute racemic or other mixtures thereof. Additional non-limiting examples of trivalent linker synthons suitable for providing trivalent linkers are illustrated in FIG. 1G.

In the exemplary binding substrates of FIGS. 1A-1F, R¹ represents a hydrophobic group, such as one of the hydrophobic groups discussed above in connection with the hydrophobic moiety. In some embodiments, R¹ is a long chain (e.g., having from 8-30 carbon atoms) saturated or unsaturated alkyl. In some embodiments, R¹ corresponds to an alkyl chain of a naturally occurring fatty acid, such as one of the fatty acids provided in Table 1, supra.

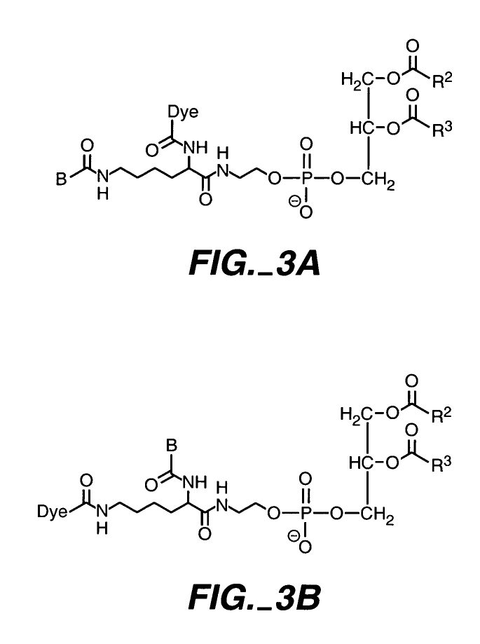

However, the binding substrates described herein need not be limited to compounds including a single hydrophobic “chain.” In some embodiments, the hydrophobic moiety of the binding substrates will include two, or even more “chains.” For example, in some embodiments, the hydrophobic moiety is provided by a phospholipid, such as a glycerophospholipid or a sphingolipid. In such embodiments, the phospholipid can be covalently linked to the remainder of the binding substrate via its polar head group, although other linkages are possible. As a specific example, the R¹—NH— group of the binding substrates illustrated in FIGS. 1A and 1B can be provided by the glycerophospholipid phosphatidyl ethanolamine. Specific embodiments of such phospholipid binding substrates are illustrated in FIGS. 3A and 3B. In FIGS. 3A and 3B, R² and R³ can be any of the previously-described hydrophobic groups, and in some embodiments correspond to the alkyl moieties of the fatty acid chains of a naturally occurring phospholipid. Moreover, although the exemplary phospholipid binding substrates include a lysine trivalent linker, any trivalent linker could be used. Binding substrates including phospholipid hydrophobic moieties can be incorporated into liposome micelles and the liposomes used in the various methods described herein.

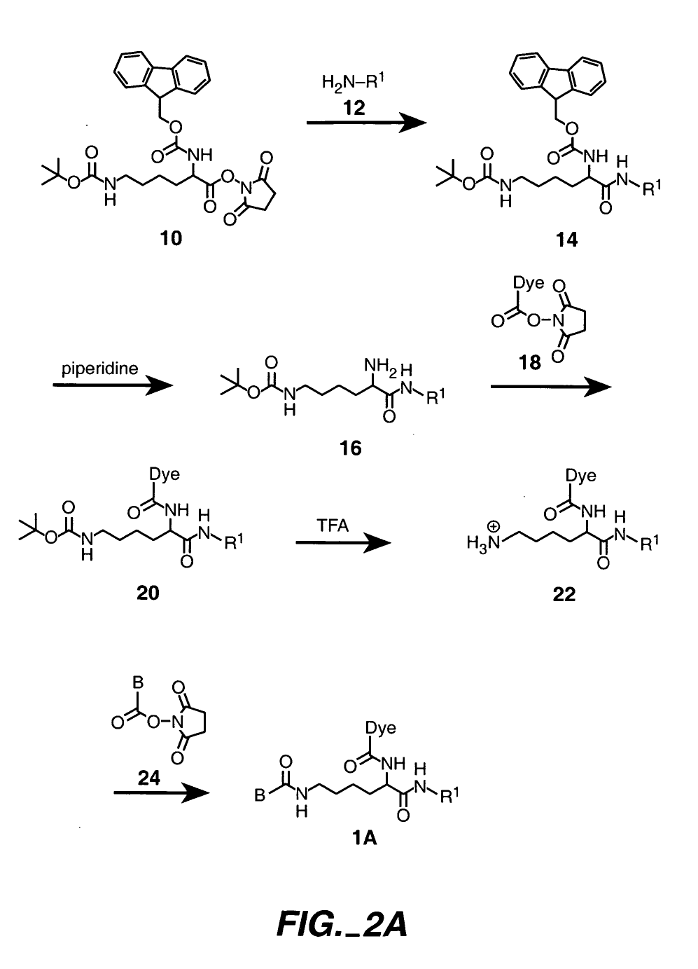

The binding substrates described herein can be readily formed by synthetic methods known in the art. An exemplary route suitable for synthesizing the substrate illustrated in FIG. 1A is provided in FIG. 2A. Referring to FIG. 2A, protected lysine NHS-ester 10 is reacted with amine 12 to yield protected compound 14. Removal of the FMOC group protecting the alpha amino group of compound 14 (for example with 30% piperidine in DMF) yields compound 16, which can be condensed with NHS-ester 18 to yield compound 20. Removal of the t-BOC group protecting the side chain (epsilon) amino group of compound 20 (for example by treatment with 1% TFA in methylene chloride for 10 minutes) yields compound 22, which can be condensed with NHS-ester 24 to yield binding substrate 1A.

The various illustrated NHS-esters may be preformed, isolated and purified, or, alternatively, they may be formed in situ by reacting the corresponding carboxylic acid with the amine in the presence of some combination of: (1) a carbodiimide reagent, e.g. dicyclohexylcarbodiimide, diisopropylcarbodiimide, or a uronium reagent, e.g. TSTU (O—(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N′,N′-tetramethyluronium hexafluorophosphate); (2) an activator, such as 1-hydroxybenzotriazole (HOBt) or 1-hydroxyazabenotriazole (HOAt); and (3) N-hydroxysuccinimide to give the NHS ester of the carboxylic acid.

Other activating and coupling reagents that could be used include TBTU (2-(1H-benzotriazo-1-yl)-1-1,3,3-tetramethyluronium hexafluorophosphate), TFFH (N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and arylsulfonyl halides, e.g. triisopropylbenzenesulfonyl chloride.

As will be appreciated by skilled artisans, activated esters and protecting groups other than those illustrated may also be employed. Suitable groups and chemistries include those conventionally employed in the solution phase and solid phase synthesis of peptides, such as the various groups and chemistries described, for example, in Lloyd-Williams et al., CHEMICAL APPROACHES TO THE SYNTHESIS OF PEPTIDES AND PROTEINS, CRC Press, 1997 and Atherton & Sheppard, SOLID PHASE PEPTIDE SYNTHESIS: A PRACTICAL APPROACH, IRL Press, 1989.

Suitably protected trivalent linker synthons, such as protected trivalent linker 18 of FIG. 2A, can be prepared using standard techniques. Methods for preparing protected amino acids that include orthogonal or non-orthogonal protection strategies are taught in the above references. Many suitably protected amino acids can also be purchased commercially. Protection strategies and chemistries for trivalent linker synthons including functional groups other than those found in amino acids are taught in standard texts, such as, for example, in Greene & Wuts, PROTECTIVE GROUPS IN ORGANIC SYNTHESIS, Second Edition, John Wiley & Sons, Inc., 1991.

Fluorescent dyes corresponding in structure to compound 18 of FIG. 2A can be prepared synthetically using conventional methods or purchased commercially (e.g., Sigma-Aldrich and/or Molecular Probes). Xanthene fluorophores, including rhodamines, fluoresceins and rhodols, are reasonably stable to the various acids and bases used to remove protecting groups such as tBOC and FMOC.

Non-limiting examples of methods that can be used to synthesize suitably reactive fluorescein and/or rhodamine dyes can be found in the various patents and publications discussed above in connection with the fluorescent moiety. Non-limiting examples of suitably reactive fluorescent dyes that are commercially available from Molecular Probes (Eugene, Oreg.) are provided in Table 3, below.

TABLE 3

| Catalog | |
|---|---|
| Number | Product Name |
| C-20050 | 5-carboxyfluorescein-bis-(5-carboxymethoxy-2-nitrobenzyl) |
| | ether, -alanine-carboxamide, succinimidyl ester (CMNB- |
| | caged carboxyfluorescein, SE) |
| C-2210 | 5-carboxyfluorescein, succinimidyl ester (5-FAM, SE) |
| C-1311 | 5-(and-6)-carboxyfluorescein, succinimidyl ester (5(6)-FAM, |
| | SE) |
| D-16 | 5-(4,6-dichlorotriazinyl) aminofluorescein (5-DTAF) |
| F-6106 | 6-(fluorescein-5-carboxamido)hexanoic acid, succinimidyl |
| | ester (5-SFX) |
| F-2182 | 6-(fluorescein-5-(and-6)-carboxamido) hexanoic acid, |
| | succinimidyl ester (5(6)-SFX) |
| F-6129 | 6-(fluorescein-5-(and-6)-carboxamido) hexanoic acid, |
| | succinimidyl ester (5(6)-SFX) |
| F-6130 | fluorescein-5-EX, succinimidyl ester |
| F-143 | fluorescein-5-isothiocyanate (FITC ‘Isomer I’) |
| F-1906 | fluorescein-5-isothiocyanate (FITC ‘Isomer I’) |
| F-1907 | fluorescein-5-isothiocyanate (FITC ‘Isomer I’) |
| F-144 | fluorescein-6-isothiocyanate (FITC ‘Isomer II’) |
| T-353 | Texas Red ® sulfonyl chloride |
| T-1905 | Texas Red ® sulfonyl chloride |
| T-10125 | Texas Red ®-X, STP ester, sodium salt |
| T-6134 | Texas Red ®-X, succinimidyl ester |
| T-20175 | Texas Red ®-X, succinimidyl ester |

Phospholipids useful for synthesizing phospholipid binding substrates (e.g., the binding substrates of FIGS. 3A and 3B) can be prepared using conventional synthetic methods, extracted from natural sources (e.g., from egg yolk, brain or plant sources) or purchased commercially (e.g., from Sigma-Aldrich and/or Avanti Polar Lipids). The synthesis of phospholipids is described in PHOSPHOLIPIDS HANDBOOK (G. Cevc, ed., Marcel Dekker (1993)), BIOCONJUGATE TECHNIQUES (G. Hermanson, Academic Press (1996)), and Subramanian et al., ARKIVOC VII:116-125 (2002). As a specific example, glycerophospholipids can be prepared from the reaction of a 3-substituted phosphoglycerol compound with selected fatty acid anhydrides. Examples of suitable phosphoglycerol compounds include glycero-3-phosphoethanolamine and glycerol-3-phosphoserine, either of which can be obtained commercially (e.g. from Sigma-Aldrich). Fatty acid anhydrides can be prepared from fatty acids, which in turn can be synthesized by conventional methods, extracted from natural sources, or purchased commercially.

Non-limiting examples of glycerophospholipids that are commercially available from Avanti Polar Lipids (Alabaster, Ala.) that can be used to prepare phospholipid binding substrates are provided in Table 4, below.

TABLE 4

| | | Avanti Catalog |
|---|---|---|
| Product Acyl Composition | M.W. | Number |
| Phosphatidylethanolamine 16:0 | 691.97 | 850705 |
| Phosphatidylethanolamine 18:1 | 744.05 | 850725 |
| N-Caproylamine-PE 16:0 | 805.13 | 870125 |
| N-Caproylamine-PE 18:1 | 857.21 | 870122 |
| N-Dodecanylamin-PE 16:0 | 889.29 | 870140 |
| N-Dodecanylamin-PE 18:1 | 941.37 | 870142 |
| Phosphatidylthio-ethanol 16:0 | 731.00 | 870160 |
| N-MCC-PE 16:0 | 928.24 | 780200 |
| N-MCC-PE 18:1 | 980.32 | 780201 |
| N-MPB-PE 16:0 | 955.20 | 870013 |
| N-MPB-PE 18:1 | 1,007.27 | 870012 |
| N-PDP-PE 16:0 | 911.22 | 870205 |
| N-PDP-PE 18:1 | 963.30 | 870202 |
| N-Succinyl-PE 16:0 | 814.03 | 870225 |
| N-Succinyl-PE 18:1 | 866.10 | 870222 |
| N-Glutaryl-PE 16:0 | 828.05 | 870245 |
| N-Glutaryl-PE 18:1 | 880.13 | 870242 |
| N-Dodecanyl-PE 16:0 | 926.24 | 870265 |
| N-Dodecanyl-PE 18:1 | 978.32 | 870262 |
| N-Biotinyl-PE 16:0 | 940.25 | 870285 |
| N-Biotinyl-PE 18:1 | 992.32 | 870282 |
| N-Biotinyl Cap-PE 16:0 | 1,053.40 | 870277 |
| N-Biotinyl Cap-PE 18:1 | 1,105.48 | 870273 |
| Phosphatidyl (Ethylene Glycol)16:0 | 714.94 | 870305 |
| Phosphatidyl (Ethylene Glycol)18:1 | 767.01 | 870302 |
| Diolylphosphatidyl serine 18:1 | 818.04 | 830035 |

In Table 4, N-MCC—PE 16:0 refers to 1,2-Dipalmitoyl-sn-glycero-3-phosphoethanolamine-N-[4-(p-maleimidomethyl)cyclohexane-carboxamide]; 16:0 MPB PE refers to 1,2-Dipalmitoyl-sn-Glycero-3-Phosphoethanolamine-N-[4-(p-maleimidophenyl)butyramide] (sodium salt); and 16:0 PDP PE refers to 1,2-Dipalmitoyl-sn-Glycero-3-Phosphoethanolamine-N-[3-(2-pyridyldithio)propionate] (sodium salt).

### 5.2.2 Quenching Molecules

Although not required for operation, in some embodiments the composition includes a quenching molecule that functions to aid the quenching of the fluorescent moieties. The quenching molecule comprises a hydrophobic moiety that is capable of integrating the quenching molecule into a micelle, such as one of the hydrophobic moieties discussed above, and a quenching moiety. The quenching moiety is selected such that it is capable of quenching the fluorescence of the fluorescent moiety of the binding substrate. If a plurality of different binding substrates are used, a quenching moiety capable of quenching the fluorescence of all or a subset of the fluorescent moieties may be selected.

Compounds capable of quenching the fluorescence of the various different types of fluorescent dyes discussed above, such as xanthene, fluorescein, rhodamine, cyanine, pthalocyanine and squaraine dyes, are well-known. Such quenching compounds can be non-fluorescent (also referred to as “dark quenchers” or “black hole quenchers,” such as those commercially available from Epoch Biosciences or Biosearch) or, alternatively, they may themselves be fluorescent. Examples of suitable non-fluorescent dark quenchers that can comprise the quenching moiety include, but are not limited to, Dabcyl, the various non-fluorescent quenchers described in U.S. Pat. No. 6.080,868 (Lee et al.) and the various non-fluorescent quenchers described in WO 03/019145 (Ewing et al.). Examples of suitable fluorescent quenchers include, but are not limited to, the various fluorescent dyes described above. In some embodiments in which the quenching moiety comprises a fluorescent dye, the fluorescence of the quenching moiety can be used as a secondary label, for example, to “track” the micelles.

The ability of a quenching moiety to quench the fluorescence of a particular fluorescent moiety may depend upon a variety of different factors, such as the mechanism(s) of action by which the quenching occurs. The mechanism of the quenching is not critical to success, and may occur, for example, by orbital overlap, by collision, by FRET, by another mechanisms or by a combination of mechanisms. The selection of a quenching moiety suitable for a particular application can be readily determined empirically. As a specific example, the dark quencher Dabcyl and the fluorescent quencher TAMRA have been shown to effectively quench the fluorescence of a variety of different fluorophores. In some embodiments, a quenching moiety can be selected based upon its spectral overlap properties with the fluorescent moiety. For example, a quenching moiety can be selected that has an absorbance spectrum that sufficiently overlaps the emission spectrum of the fluorescent moiety of a binding substrate such that the quenching moiety quenches the fluorescence of the fluorescent moiety when in close proximity thereto.

### 5.2.3 Micelles Comprising Binding Substrates and/or Quenching Molecules

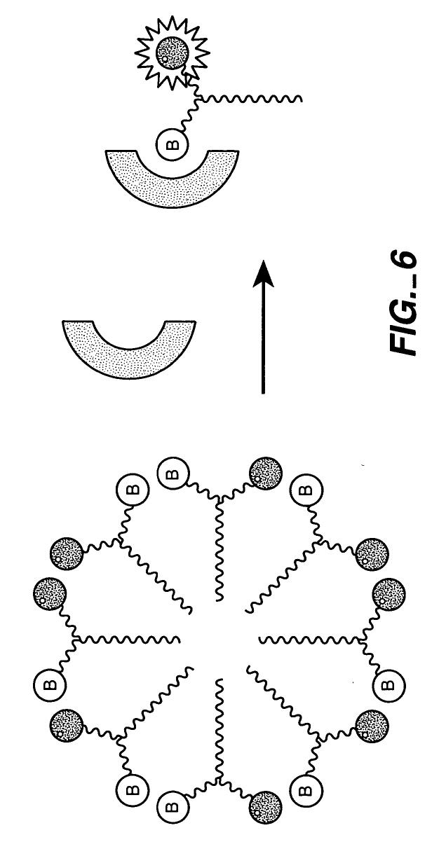

While not intending to be bound by any theory of operation, it is believed that when the binding substrate is dispersed in an aqueous solvent at a concentration above a threshold level in the absence of a binding partner for the binding moiety, it aggregates into micelles. As illustrated in FIG. 6, the fluorescent moieties on binding substrates in the same micelle have a high local concentration and close proximity, which results in quenching of their fluorescent signals. Binding of the binding moiety by another molecule reduces or eliminates the quenching effect, leading to an increase in the fluorescence signal of the fluorescent moiety. Although the mechanism by which the quenching effect is reduced or eliminated is unknown and not critical for success, it is believed that binding of the binding moiety causes the micelle to disintegrate or, alternatively, causes the bound binding substrates to be removed from the micelle, thereby separating (by diffusion into the surrounding solution) the bound binding substrates from the remaining micellar fluorescent moieties and/or quenching moieties, so that a fluorescent signal from the bound binding substrates can be more easily detected.

Accordingly, the present disclosure also concerns micelles comprising the binding substrates described herein, wherein the fluorescence of the fluorescent moieties on the binding substrates is quenched, and in some embodiments substantially quenched. Depending upon the mechanism by which the quenching effect is achieved (e.g., whether by self-quenching or with the aid of a quenching molecule), the binding substrate can comprise a primary component or constituent of the micelle or, alternatively, the binding substrate can comprise a minor component or constituent of the micelle. The form of the micelle is not critical to success. The micelle can range in form from a “detergent-like” micelle which does not enclose a part of the aqueous solvent (such as the micelle illustrated in FIG. 6) to a “vesicle-like” micelle which encloses a part of the aqueous solvent. Such “vesicle-like” micelles can be small or large in size, and although they can be unilamellar or multilamellar, unilamellar vesicle-like micelles are preferred. The micelle can also take on any type of three-dimensional shape or structure, including, for example, spherical, oblate, discoidal or cubic.

The micelles can be formed in situ during the course of an assay, or they can be preformed and added to an assay in micellar form. Micelles formed in situ can be prepared by mixing the binding substrate and any optional quenching molecules or other components comprising the micelle in the assay buffer at concentrations at or above their critical micelle concentrations. The assay buffer can be optionally agitated to promote micelle formation.

The binding substrate and optional quenching molecule should be included in the micelle at molar ratios that permit them to perform their respective functions. For example, the binding substrate should be included at a molar ratio that provides a sufficient number of binding moieties such that binding between the binding moiety and another molecule is likely to occur. The optional quenching molecule should be included at a molar ratio that yields an acceptable dynamic range of fluorescence signal under the assay conditions. For example, the binding substrates and optional quenching molecule can be included in the micelles at molar ratios sufficient to provide quenching of the fluorescent moieties in the micelle and a detectable increase in fluorescence over this quenched background when the micelle is bound by a binding partner for the binding moiety. Embodiments in which the quenching effect is achieved by self-quenching of the fluorescent moieties without the aid of quenching molecules may require a higher molar ratio of binding substrates than embodiments employing quenching molecules.

For any particular micellar form and desired binding substrate and optional quenching molecule, suitable molar ratios can be determined empirically. For example, the appropriate amount of binding substrate and optional quenching molecule to include can be determined by preparing several batches of micelles comprising varying molar ratios of binding substrates and optional quenching molecules and comparing the increase in fluorescence observed upon binding with a known binding partner for the binding moiety. As will be appreciated, other methods could also be used to empirically determine optimal molar ratios of binding substrates and optional quenching molecules for particular applications.

In some embodiments, the micelle is a “detergent like” micelle that is wholly composed of binding substrate(s).

In other embodiments, the micelle is a liposome. A liposome is a self-closed vesicle where one or several lipid membranes encapsulate part of the solvent. The composition and form of these lipid vesicles are analogous to that of cell membranes with hydrophilic polar groups directed inward and outward toward the aqueous media and hydrophobic fatty acids intercalated within the bilayer. Liposomes are formed when thin lipid films or lipid cakes are hydrated and stacks of liquid crystalline bilayers become fluid and swell. Liposomes may be unilamellar and/or multilamellar. Unilamellar liposome vesicles are typically classified as small (SUTs) (less than 50 nm in diameter), large (LUVs) approx. 50-250 nm in diameter) or giant (approx. 1 micron in diameter). Small (SMV) and large, multilamellar liposome vesicles (LMV) can also be formed. Multilamellar liposomes are classically described as having concentric bilayers, an “onion morphology.” A type of multilamellar liposome termed oligolamellar liposomes are typically described as multilamellar liposomes which have increased aqueous space between bilayers or which have liposomes nested within bilayers in a nonconcentric fashion. Once these complexes have formed, reducing the size of the complex usually requires energy input in the form of sonic energy (sonication) or mechanical energy (extrusion).

Liposomes are typically comprised of phospholipids having hydrophobic tails or other bulky hydrophobic moieties that disfavor the formation of detergent-like micelles. Liposomes can be formed from any single type of phospholipids or mixture of phospholipids. A liposome preparation can include one or more of phosphatidic acid, phosphatidylethanolamine, phosphatidylcholine, phosphatidylinositols, phosphatidylglycerol, sphingomylelin, cardiolipin, lecithin, phosphatidylserine, cephalin, cerebrosides, dicetylphosphate, steroids, terpenes, stearylamine, dodecylamine, hexadecylamine, acetylpalmitate, glycerol ricinoleate, hexadecyl stearate, isopropyl myristate, dioctadecylammonium bromide, amphoteric polymers, triethanolamine lauryl sulfate and cationic lipids, 1-alkyl-2-acyl-phosphoglycerides, and 1-alkyl-1-enyl-2-acyl-phosphoglycerides. Other lipids useful in forming liposomes include cationic lipids, examples of which include dioctadecyl dimethyl ammonium bromide/chloride (DODAB/C) and dioleoyloxy-3-(trimethylammonio)propane (DOTAP). See, for example, Lasic, LIPOSOMES IN GENE DELIVERY, CRC Press, New York, pp. 81-86 (1997). Cholesterols may also be used.

A wide variety of suitable lipids are commercially available (such as from Avanti Polar Lipids, Inc. Alabaster, Ala.). Liposome kits are commercially available (e.g. from Boehringer-Mannheim, ProMega, and Life Technologies (Gibco)). Non-limiting examples of suitable lipids include 1,2-dimyristoyl-sn-glycero-3-phosphate (Monosodium Salt) (DMPA.Na) (Avanti catalog no. 830845), 1,2-dimyristoyl-sn-glycero-3-phosphate (Monosodium Salt) (DOPS.Na) (Avanti catalog no. 830035), and 1,2-dioleoyl-3-trimethylammonium-propane (Chloride Salt) (DTOAP.Cl) (Avanti catalog no. 890890).

Liposomes can also include synthetic lipid compounds such as D-erythro (C-18) derivatives including sphingosine, ceramide derivatives, and sphinganine; glycosylated (C18) sphingosine and phospholipid derivatives; D-erythro (C17) derivatives; D-erythro (C20) derivatives; and L-threo (C18) derivatives, all of which are commercially available (Avanti).

Liposomes can include or be formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally includes an ester moiety (ester of a fatty acid), the ester linkage can be replaced with an ether linkage at position 1 and/or 2.

In some embodiments, binding substrate-containing liposomes which may be useful include, in addition to the binding substrate, lipids such as phosphatidylcholine (PC) and phosphatidylethanolamine (PE). Preferably, phosphatidylcholine ranges from about 50 to 95 mol percent of the total lipid content of the liposome, and phosphatidylethanolamine ranges from 2 to 20 mol percent. More preferably, phosphatidylcholine ranges from about 60 to 90 mol percent, and phosphatidylethanolamine ranges from about 4 to 12 mol percent.

The liposomes may include cholesterol. Cholesterol can intercalate within the liposome bilayer by occupying the regions created by the bulky phospholipid head groups. This increases the packing density and structural stability of the bilayer (New, R.R.C. (ed): LIPOSOMES: A PRACTICAL APPROACH, Oxford University Press, New York, pp 19-21 (1990)). Cholesterol also affect the fluidity and permeability of the membrane. The concentration of cholesterol in liposomes can range, for example, from about 5 to about 60 mol percent.

The composition of liposomes comprising binding substrates can be selected based an a variety of factors including cost, transition temperature of the lipids, stability during storage, and stability of the liposomes under the reaction conditions, as well as the presence of phospholipases in the sample being assayed.

Properties of liposomes can vary depending on the composition (cationic, anionic, neutral lipid species). However, the same preparation method may be used for all lipid vesicles regardless of composition. The general elements of the procedure involve preparation of the lipid for hydration, hydration with agitation, and sizing to a homogeneous distribution of vesicles.

Liposomes including binding substrates can be prepared using conventional methods, such as described in Lasic, LIPOSOMES IN GENE DELIVERY, CRC Press, New York, pp.67-112 (1997); ANN. REV. BIOPHYS. BIOENG. 9:467-508 (1980); U.S. Pat. Nos. 4,229,360, 4,235,871, 4,241,046, 6,458,381 and 6,534,018. When preparing liposomes with mixed lipid composition, the lipids can first be dissolved and mixed in an organic solvent to assure a homogeneous mixture of lipids. Usually this process is carried out using chloroform or chloroform:methanol mixtures. Typically lipid solutions can be prepared at 10-20 mg lipid/ml organic solvent, although higher concentrations may be used if the lipid solubility and mixing are acceptable. Once the lipids are thoroughly mixed in the organic solvent, the solvent is removed to yield a lipid film. For small volumes of organic solvent (<1 ml), the solvent may be evaporated using a dry nitrogen or argon stream in a fume hood. For larger volumes, the organic solvent can be removed by rotary evaporation yielding a thin lipid film on the sides of a round bottom flask. The lipid film is thoroughly dried to remove residual organic solvent by placing the vial or flask on a vacuum pump overnight. If the use of chloroform is objectionable, tertiary butanol, cyclohexane or other alternatives can be used to dissolve the lipid(s). The lipid solution is transferred to containers and frozen by placing the containers on a block of dry ice or swirling the container in a dry ice-acetone or alcohol (ethanol or methanol) bath. Care should be taken when using the bath procedure that the container can withstand sudden temperature changes without cracking. After freezing completely, the frozen lipid cake is placed on a vacuum pump and lyophilized until dry (1-3 days depending on volume). The thickness of the lipid cake preferably is no more than the diameter of the container being used for lyophilization. Dry lipid films or cakes can be removed from the vacuum pump, the container close tightly and taped, and stored frozen until ready to hydrate.

Hydration of the dry lipid film/cake is accomplished simply by adding an aqueous medium to the container of dry lipid and agitating. The temperature of the hydrating medium should be above the gel-liquid crystal transition temperature (Tc) of the lipid that has the highest Tc. After addition of the hydrating medium, the lipid suspension is maintained above the Tc during the hydration period. For high transition lipids, this is easily accomplished by transferring the lipid suspension to a round bottom flask and placing the flask on a rotary evaporation system without a vacuum. Spinning the round bottom flask in the warm water bath maintained at a temperature above the Tc of the lipid suspension allows the lipid to hydrate in its fluid phase with adequate agitation. Hydration time may differ slightly among lipid species and structure. A hydration time of 1 hour with vigorous shaking, mixing, or stirring is recommended. It is also believed that allowing the vesicle suspension to stand overnight (aging) prior to downsizing may make the sizing process easier and improves the homogeneity of the size distribution. The hydration medium is generally determined by the application of the lipid vesicles. Suitable hydration media include distilled water, buffer solutions, saline, and nonelectrolytes such as sugar solutions. During hydration some lipids form complexes unique to their structure. Highly charged lipids have been observed to form a viscous gel when hydrated with low ionic strength solutions. The gel formation can be alleviated by addition of salt or by downsizing the lipid suspension. The product of hydration usually is a large, multilamellar vesicle (LMV) analogous in structure to an onion, with each lipid bilayer separated by a water layer. LMV can be directly used in the compositions and methods described herein. LMV can also be further downsized by a variety of techniques, including sonication or extrusion.

Disruption of LMV suspensions using sonic energy (sonication) typically produces small, unilamellar vesicles (SUV) with diameters in the range of 15-50 nm. Instrumentation for preparation of sonicated particles includes bath, probe tip and cup-horn sonicators. Sonication of an LMV dispersion is accomplished by placing a test tube containing the suspension in a bath sonicator (or placing the tip of the sonicator in the test tube) and sonicating for 5-10 minutes above the Tc of the lipid. The lipid suspension should begin to clarify to yield a slightly hazy transparent solution. The haze is due to light scattering induced by residual large particles remaining in the suspension. These particles can be removed by centrifugation to yield a clear suspension of SUV. Mean size and distribution is influenced by composition and concentration, temperature, sonication time and power, volume, and sonicator tuning.

An alternative method for sizing is extrusion. Lipid extrusion is a technique in which a lipid suspension is forced through a polycarbonate filter with a defined pore size to yield particles having a diameter near the pore size of the filter used. Prior to extrusion through the final pore size, LMV suspensions can be disrupted either by several freeze-thaw cycles or by prefiltering the suspension through a larger pore size (typically 0.2-1.0 μm). This method helps prevent the membranes from fouling and improves the homogeneity of the size distribution of the final suspension. As with all procedures for downsizing LMV dispersions, the extrusion preferably is done at a temperature above the Tc of the lipid. Extrusion through filters with 100 nm pores typically yields large, unilamellar vesicles (LUV) with a mean diameter of 120-140 nm. Mean particle size also depends on lipid composition and is reproducible from batch to batch.

Preparations of liposomes containing binding substrates can include stabilizing agents, such as, for example, antioxidants, such as α-tocopherol and chelators. Other agents, including disaccharides, ascorbic acid, cysteine, monothioglycerol, sodium bisulfite, sodium metabisulfite, gentisic acid, and inositol, may also be used. For a non-limiting list of agents that can be included in a liposome preparation, as well as their useful concentration ranges, see LIPOSOMES, 2d edition, Torchillin and Weissig, Eds, Oxford University Press, NY (2003). The liposomes can be lyophilized for storage and/or for inclusion in kits.

The micelles can include more than one type of binding substrate and/or optional quenching molecule. For example, a micelle can include two different types of binding substrates. An observed increase in the fluorescence signal in a binding assay carried out with this type of micelle indicates that one or both of the binding moieties bound a molecule(s) in the sample.

In embodiments that utilize more than one binding substrate, the fluorescent moieties on the different binding substrates can be selected such that their fluorescence signals are spectrally resolvable. In this manner, the different binding moieties comprising the micelle can be correlated to different colored signals. A change in fluorescence signals at a specified wavelength can indicate not only that the micelle bound the molecule(s) in the sample, but also which binding moiety bound.

Micelles that are vesicle-like, such as liposomes, can optionally encapsulate agents within their interior. In some embodiments, the liposome can encapsulate a fluorescent dye (or combination of dyes) which can be used as a tracer to assess the integrity of the liposomes during preparation, storage and/or subsequent use.

In some embodiments, an encapsulated agent can be selected that quenches the fluorescence of the fluorescent moieties. As discussed above in connection with quenching molecules, such quenching agents can be “dark,” or alternatively, they may themselves be fluorescent.

In those embodiments in which fluorescent dyes or quenching agents are encapsulated within the micelle, conventional methods can be used for loading, such as reverse phase methods and sonication (e.g. Lasic, LIPOSOMES IN GENE DELIVERY, CRC Press, New York, p.93 (1997); and U.S. Pat. No. 4,888,288).

### 5.3 Methods

The binding substrates and micelles described herein can be used in a variety of different assays, such as, for example, to characterize, identify, detect, quantify and/or screen for binding partners for the binding moiety and/or inhibitors of the binding moiety and other molecules. In some embodiments, a composition comprising a binding substrate (and/or a micelle comprising a binding substrate), and optionally a known binding partner for the biding moiety, is contacted with a sample containing or suspected of containing a known or putative binding partner for the binding moiety. The sample may then be monitored for a change in fluorescence, which correlates with the presence of the binding partner in the sample.

The binding assay typically includes a buffer, such as a buffer described in the “Biological Buffers” section of the 2000-2001 Sigma Catalog, as well as any cofactors or other agents that may be required for binding between the molecule(s) of the sample and the binding moiety. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the binding interaction of the binding molecule to be detected. For example, the pH can be from 2 to 12, from 4 to 11, or from 6 to 10. The identities and concentrations of any necessary cofactors and/or agents will depend upon the binding interaction, and will be apparent to those of skill in the art.

The binding assay typically does not require the presence of detergents or other components. In general, it is desirable to avoid high concentrations of components in the binding mixture that can adversely affect the binding interaction and/or the fluorescence properties of the bound binding substrate, or that can interfere with the analysis of modulators such as inhibitors, such as described herein below.

Depending upon the specific assay to be performed, the binding assay may include a known binding partner for the binding moiety. When included, the known binding partner may be added to the binding assay in a free, unbound form, or it may be added to the assay in the form of a pre-formed binding partner-binding substrate complex. Such complexes can be prepared by incubating the binding substrate with the a binding partner under conditions conducive to binding.

The concentrations of binding substrate (and/or micelles comprising the binding substrate), candidate compounds and/or known binding partners (if present) in the binding assay is not critical, provided that the various compounds are included at concentrations sufficient to produce a detectable change in fluorescence. Typically, the binding substrate is included in the binding assay at a concentration at or above its critical micelle concentration (“CMC”).

In some embodiments it may be desirable to utilize concentrations of candidate compounds, binding substrates and/or known binding partners that are selected based upon the desired outcome of the binding assay. For example, if the binding assay will be used to screen for and/or characterize binding partners for the binding moiety that have a Kd at or below a threshold level, it may be desirable to use a concentration of candidate compound and/or binding substrate (and/or micelles including a binding substrate) that is at or above this threshold level.

As another specific example, if the binding assay will be carried out in the presence of a known binding partner for the binding moiety and used to detect, screen for, identify and/or characterize an inhibitor of the binding moiety-binding partner pair that has a threshold Kd, the binding partner can be included in a binding assay at a concentration at or above its Kd and the candidate compound can be included in the assay at a concentration at or above the threshold Kd. Moreover, in embodiments in which it is desirable to identify relatively “weak” competitive inhibitors (e.g., those having Kds in the range of 1-10 μM), such as, for example, assays designed to screen for and/or identify drug candidates or leads, it may be desirable to utilize a known binding partner that has a Kd above the desired threshold level for the inhibitor in order to insure that the candidate inhibitory compound can compete off the known binding partner.

As another specific example, if a competitive binding assay will be used to quantify the amount of a known binding compound in a sample, the binding substrate and inhibitor can be included in the binding assay at concentrations approximately equal to the expected amount of binding compound in the sample. In some embodiments, the binding substrate and inhibitor can be included in the binding assay in equimolar amounts.

In the methods described herein, the fluorescence signal can be monitored using conventional methods and instruments. In some embodiments, a multiwavelength fluorescence detector can be utilized. The detector can be used to excite the fluorescent labels at one wavelength and detect emissions at multiple wavelengths, or excite at multiple wavelengths and detect at one emission wavelength. Alternatively, the sample can be excited using “zero-order” excitation in which the full spectrum of light (e.g., from xenon lamp) illuminates the cuvette. Each fluorescent moiety can absorb at its characteristic wavelength of light and then emit maximum fluorescence. The multiple emission signals can be monitored independently. Preferably, a suitable detector can be programmed to detect more than one excitation emission wavelength substantially simultaneously, such as that commercially available under the trade designation HP1100 (G1321A), from Hewlett Packard, Wilmington, Del. Thus, the fluorescent moiety can be detected at programmed emission wavelengths at various intervals during a reaction.

Detection of fluorescent signal can be performed in any appropriate way. Advantageously, the binding substrate compositions, micelles and/or methods can be used in a continuous monitoring phase, in real time, to allow the user to rapidly determine whether there is a binding event between the binding moiety and a binding molecule. The fluorescent signal can be measured from at least two different time points, usually before and after contacting the binding substrate and/or micelle with the sample.

Alternatively, the fluorescent signal can be measured in an end-point embodiment in which a signal is measured after a certain amount of time, and the signal is compared against a control signal (e.g., before contact with the sample), threshold signal, or standard curve.

The present teachings contemplate not only detecting binding interactions, but also methods involving: (1) screening for, identifying and/or quantifying binding compounds in a sample, (2) determining dissociation constants with respect to selected binding partners, (3) detecting, screening for, and/or characterizing binding partners for binding moieties of interests, (4) detecting, screening for, identifying and/or characterizing inhibitors, activators, and/or modulators of binding interactions, and (5) determining binding specificities and/or binding consensus sequences or binding consensus structures for selected molecules.

For example, in screening for binding activity, a sample that contains, or may contain, a known or candidate binding compound can be mixed with a binding substrate, and the fluorescence measured to determine whether an increase in fluorescence has occurred. Screening may be performed on numerous samples simultaneously in a multi-well or multi-reaction plate or device to increase the rate of throughput. The dissociation constant (Kd) of the interaction may be determined by standard methods.

In some embodiments, the assay mixture may contain two or more different candidate compounds. This may be useful, for example, to screen multiple candidates simultaneously to determine if at least one of the candidate compounds binds the binding moiety.

In other embodiments, the assay mixture may contain two or more different binding substrates. This may be useful, for example, to screen multiple binding moieties simultaneously to determine if at least one of the binding moieties binds a compound of interest in the sample.

In assays employing different binding substrates, each different substrate may be tested separately in different assay mixtures, or two or more substrates may be present simultaneously in a reaction mixture. In embodiments in which the different substrates are present simultaneously in the reaction mixture, the substrates can contain the same fluorescent moiety, in which case the observed fluorescent signal is the sum of the signals from binding with both substrates. Alternatively, the different substrates can contain different, fluorescently distinguishable fluorescent moieties that allow separate monitoring and/or detection of binding with each different substrate simultaneously in the same mixture. The fluorescent moieties can be selected such that all or a subset of them are excitable by the same excitation source, or they may be excitable by different excitation sources. They can also be selected to have additional properties, such as, for example, the ability to quench one another when in close proximity thereto, by, for example, orbital overlap, collisional quenching, FRET or another mechanism (or combination of mechanisms).

In some embodiments, assays carried out with a plurality of different binding substrates may utilize pre-formed micelles, each composed of a different binding substrate.

Detecting, screening for, identifying and/or characterizing inhibitors, activators, and/or modulators of binding interactions can be performed by forming assay mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constant), K_{H} (Hill coefficient), Kd (dissociation constant) and the like to characterize the concentration dependence of the effect that such substances have on binding activity.

### 5.4 Kits

Also provided herein are kits for performing the methods described herein. In some embodiments, the kit comprises at least one binding substrate for detecting a target binding interaction, and a buffer for preparing an assay mixture that facilitates the binding interaction. The kit may optionally include a known binding partner for the binding substrate. The buffer may be provided in a container in dry form or liquid form. The choice of a particular buffer may depend on various factors, such as the pH optimum for the binding interaction to be detected, the solubility and fluorescence properties of the fluorescent moiety in the binding substrate, and the pH of the sample from which the target binding molecule is obtained. The buffer is usually added to the assay mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the binding assay, as discussed above. The pH of the assay mixture may also be titrated with acid or base to reach a final, desired pH. The kit may additionally include other components that are beneficial to the binding interaction, such as salts (e.g., KCl, NaCl, or NaOAc), metal salts (e.g., Ca2+ salts such as CaCl₂, MgCl₂, MnCl₂, ZnCl₂, or Zn(OAc), detergents (e.g., TWEEN 20), and/or other components that may be useful for a particular binding interaction. These other components can be provided separately from each other or mixed together in dry or liquid form.

The binding substrate can also be provided in dry or liquid form, together with or separate from the buffer. To facilitate dissolution in the assay mixture, the binding substrate can be provided in an aqueous solution, partially aqueous solution, or non-aqueous stock solution that is miscible with the other components of the-assay mixture. For example, in addition to water, a substrate solution may also contain a cosolvent such as dimethyl formamide, dimethylsulfonate, methanol or ethanol, typically in a range of 1%-10% (v:v).

The binding substrate may also be provided in the form of pre-formed micelles, which may be in either dry or liquid form.

If the kit includes the optional binding partner, it may be packaged separately, or, alternatively, it may be provided as a complex with the binding substrate.

Aspects of the present teachings may be further understood in light of the following examples, which should not be construed as limiting the scope of the present teachings in anyway.

### 6. EXAMPLES

### 6.1 Example 1

### Detection of Thyroxine/Anti-Thyroxine Binding Interactions

This example demonstrates the ability to detect binding interactions between thyroxine and a thyroxine-specific monoclonal antibody using an exemplary binding substrate in which the binding moiety comprises thyroxine (Compound 107).

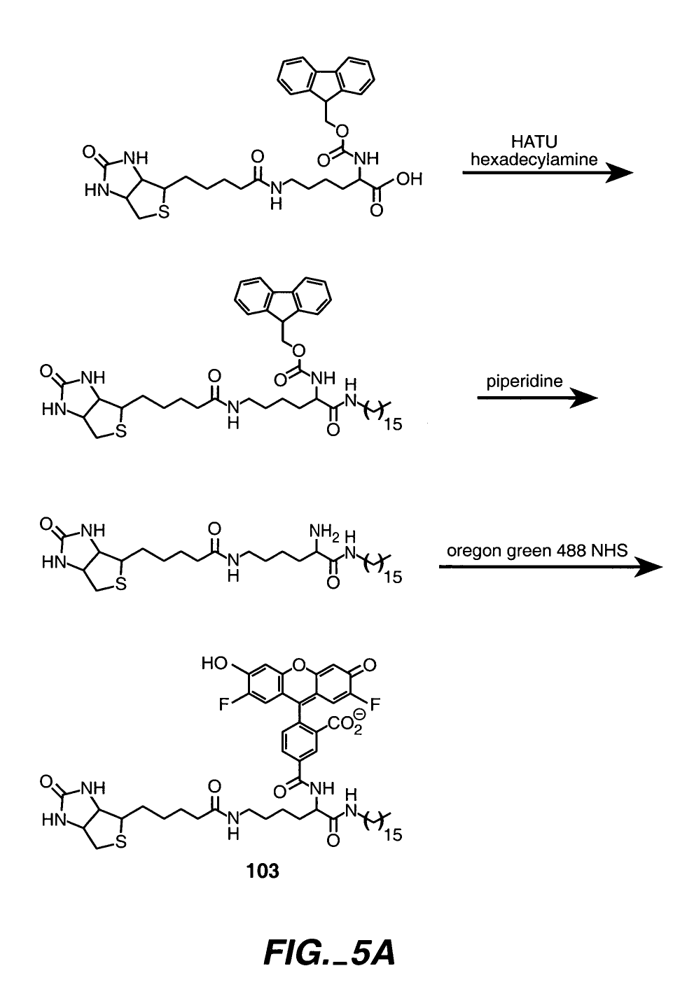

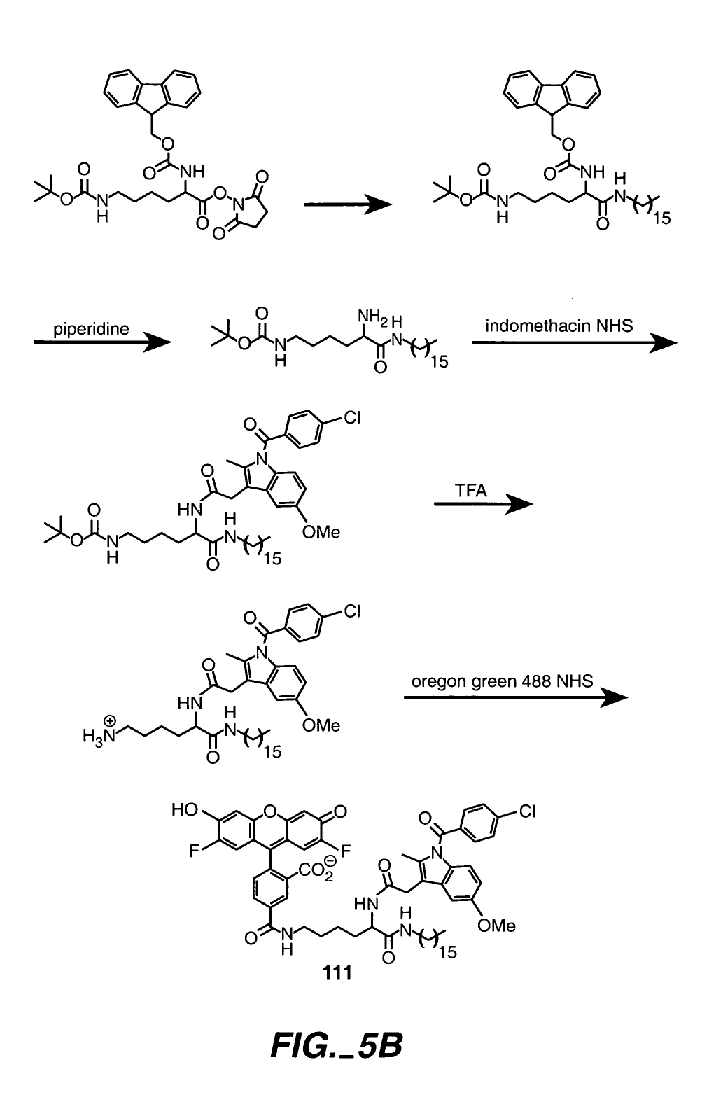

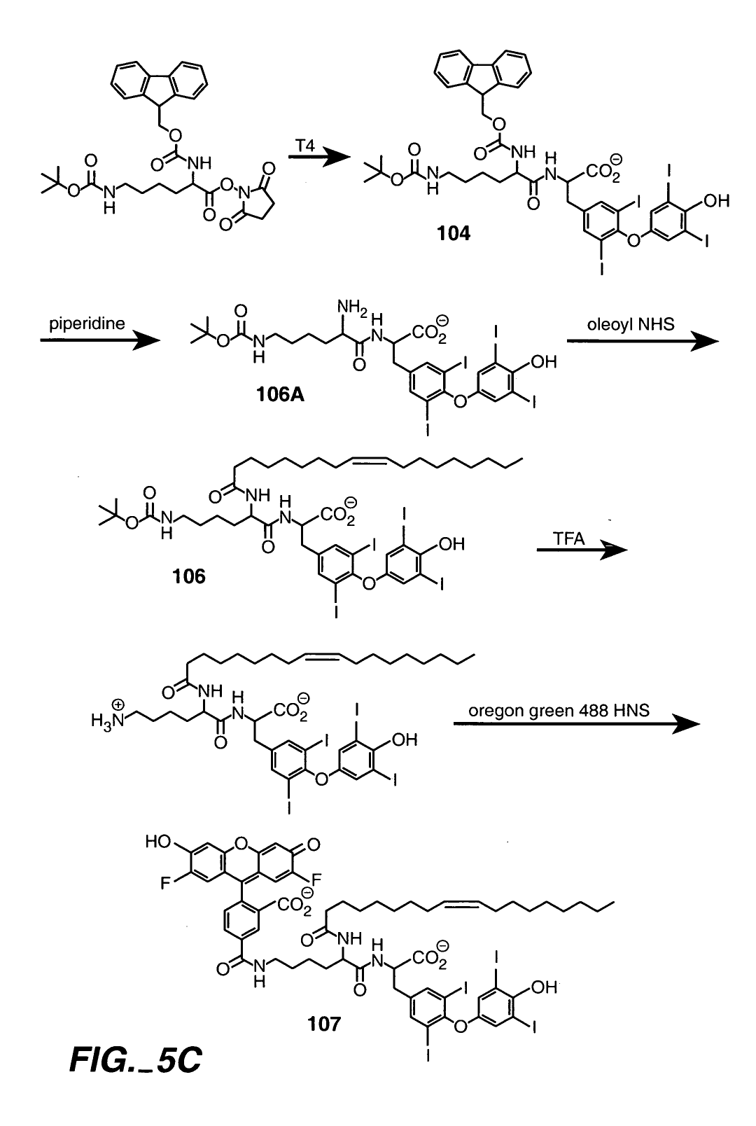

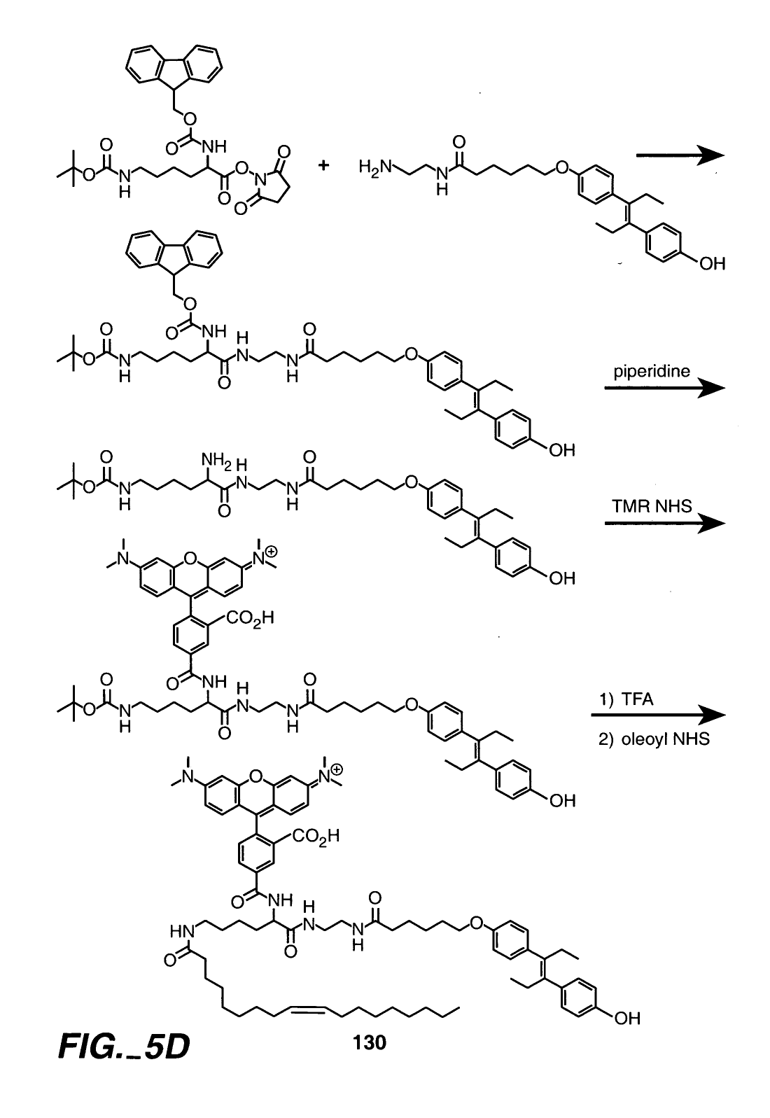

Synthesis of Compound 107. The exemplary binding substrate 107 was synthesized as follows and as illustrated in FIG. 5C.

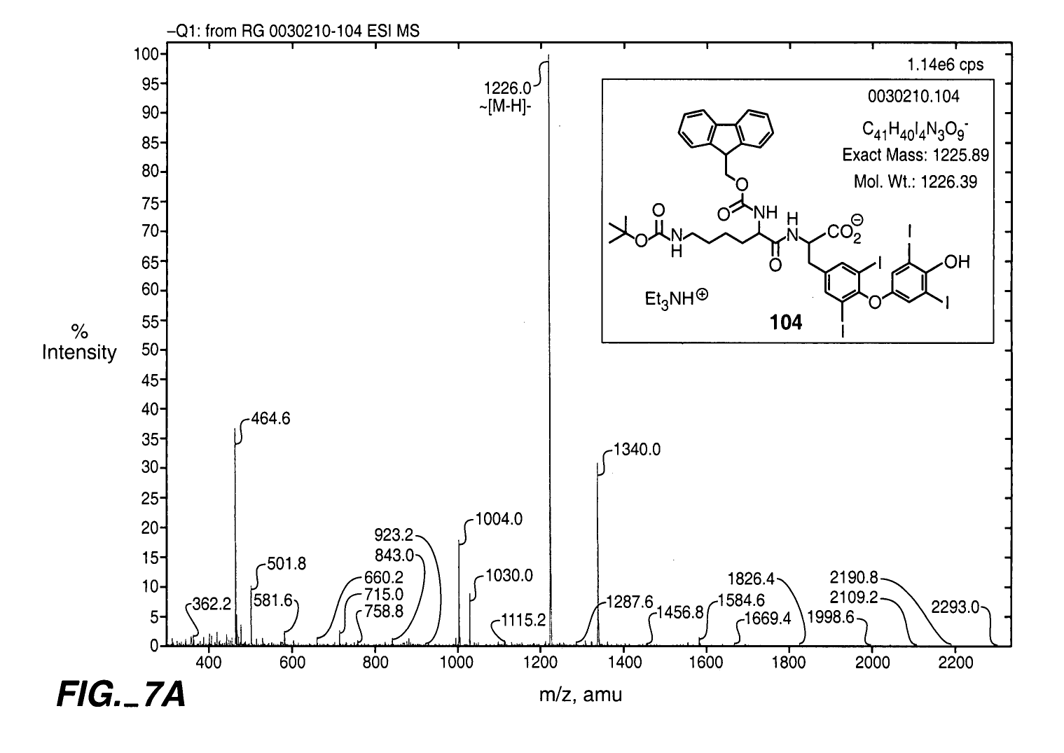

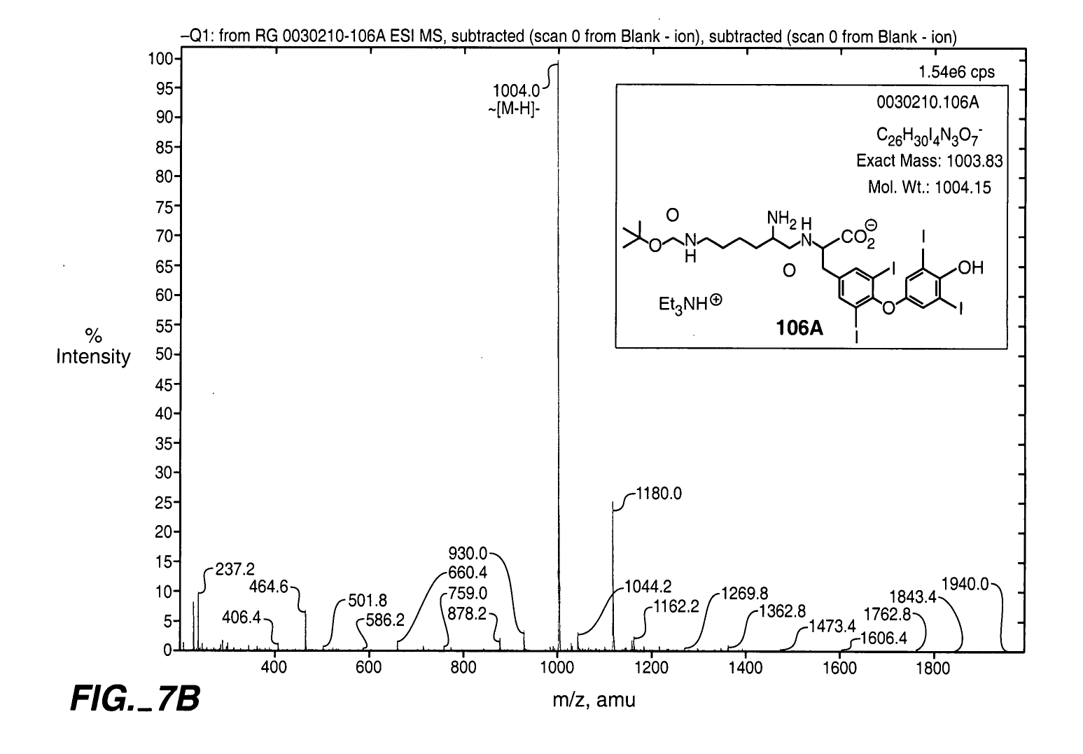

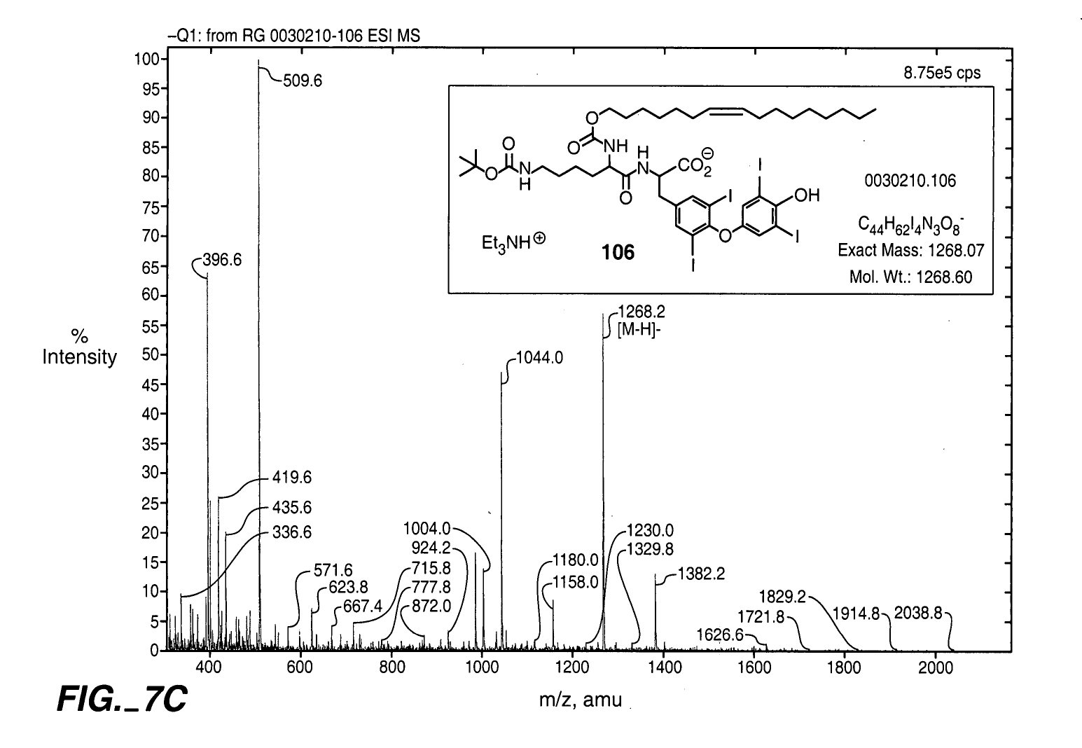

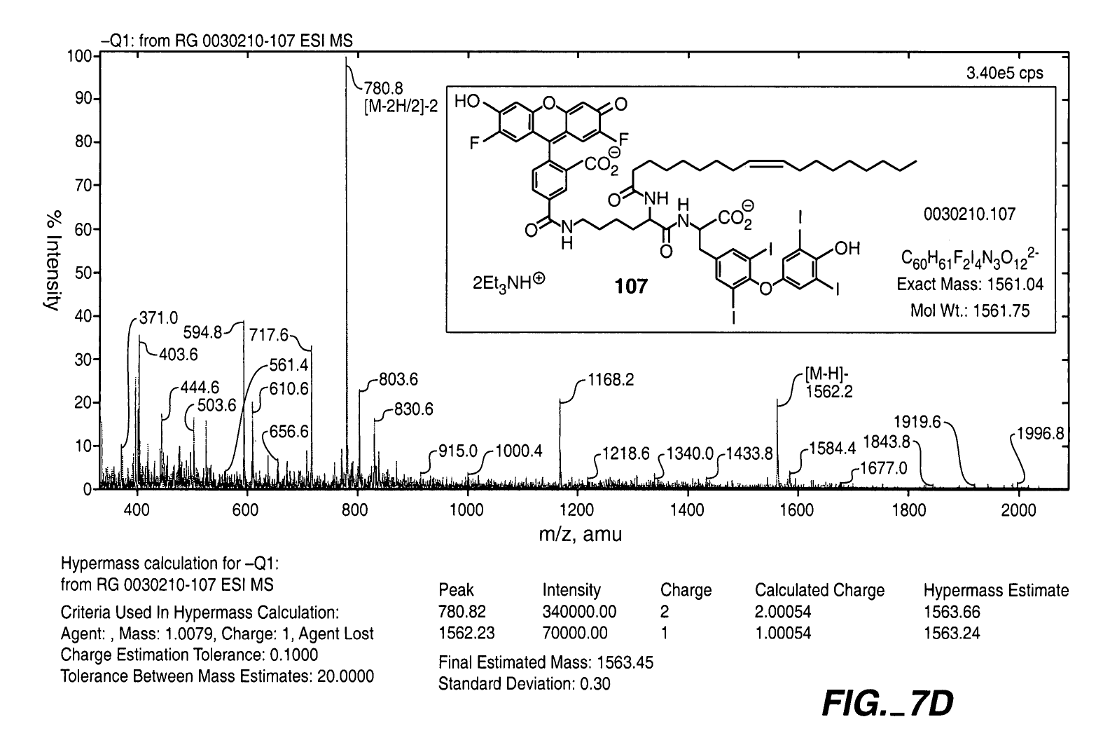

Compound 104: L-Thyroxine (T4, 69 mg, 88 μmol, Sigma part # T-1775) was dissolved in 1:1 dichloromethane (DCM)/trifluoroacetic acid (TFA) (6 ml). The solvent was evaporated to leave T4 as its DCM soluble TFA salt. DCM (5 ml) and triethylamine (TEA, 61 ml, 440 μmol) were added followed by N—(N-α-FMOC—N-ε-t-Boc-L-lysyloxy)succinimide (“FMOC-lys(BOC)-Osu”; 50 mg, 88 μmol, Novabiochem part # 04-12-1040). After 15 min the solvent was evaporated and the product was purified by silica gel (J.T. Baker part # 7024-01) chromatography (DCM/methanol, 95:5) to give a yellow solid (104, 46 mg, 40%). The electrospray ionization mass spectrum (ESI ms) was consistent with the assigned structure (see FIG. 7A).

Compound 106A: Compound 104 (46 mg, 35 μmol) was dissolved in 20% piperidine in dimethylformamide (DMF, 5 ml). After 30 min the solvent was evaporated and the product was purified by trituration with ethyl ether to leave a yellow solid (106A, 32 mg, 90%). The ESI ms was consistent with the assigned structure (see FIG. 7B).

Compound 106: Compound 106A (32 mg, 32 μmol) was dissolved in DMF (3 ml) and oleic acid N-hydroxysuccinimide ester (13 mg, 35 μmol, Sigma part # O-9506) and TEA (10 μl, 70 μmol) were added. After 15 min the solvent was evaporated and the product was purified by silica gel (J.T. Baker part # 7024-01) chromatography (DCM/methanol, 98:2) to give a yellow solid (106, 22 mg, 55%). The ESI ms was consistent with the assigned structure (see FIG. 7C).

Compound 107: Compound 106 (22 mg, 17 μmol) was dissolved in 1:1 DCM/TFA (5 ml) in order to cleave the BOC protecting group. After 1 hr the solvent was evaporated under high vacuum. DMF (2 ml), oregon green 488 carboxylic acid, succinimidyl ester *5-isomer* (10 mg, 20 μmol, molecular probes part # O-6147) and TEA (22 μl, 160 μmol) were added. After 15 min the reaction mixture was diluted with 100 mM TEAA (18 ml) and the product was purified by C18 (J.T. Baker part # 7025-01) reverse phase chromatography (methanol/100 mM triethylamine acetate (“TEAA”); 70:30) to give an orange solid (107, 8 mg, 25% for two steps). The ESI ms was consistent with the assigned structure (see FIG. 7D).

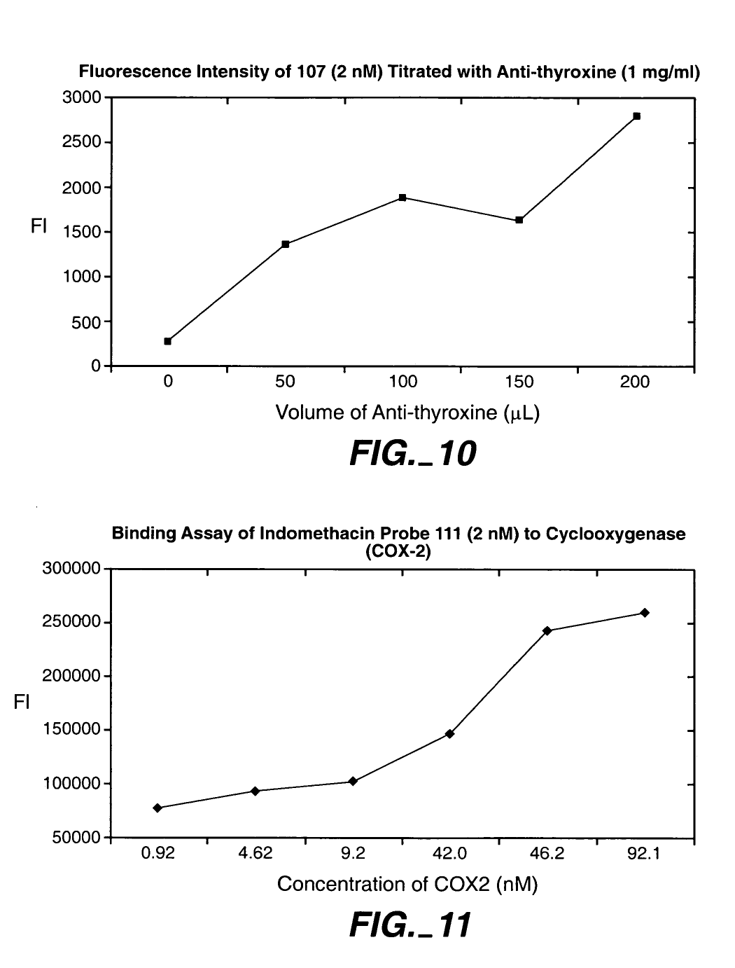

Detection of Binding. Compound 107 (2 ml, 2 nM) in phosphate-buffered saline (“PBS”; 154 mM NaCl, 1.9 mM NaH₂PO₄, 8.1 mM Na₂HPO₄, pH 7.2-7.4) was added to a 3.5 ml quartz cuvette and the fluorescence was measured with a Spex fluorolog 3 fluorimeter. Solutions of compound 107 and increasing amounts of monoclonal anti-L-thyroxine from mouse (1 mg/ml, Sigma part # T 3901) were prepared and the fluorescence was again measured (FIG. 10). The fluorescence increased about ten fold.

### 6.2 Example 2

### Detection of Indomethacin/COX-2 Binding Interactions

This example demonstrates the ability to detect binding interactions between indomethacin and an enzymatically inactive COX-2 apoenzyme using an exemplary binding substrate in which the binding moiety comprises indomethacin (Compound 111).

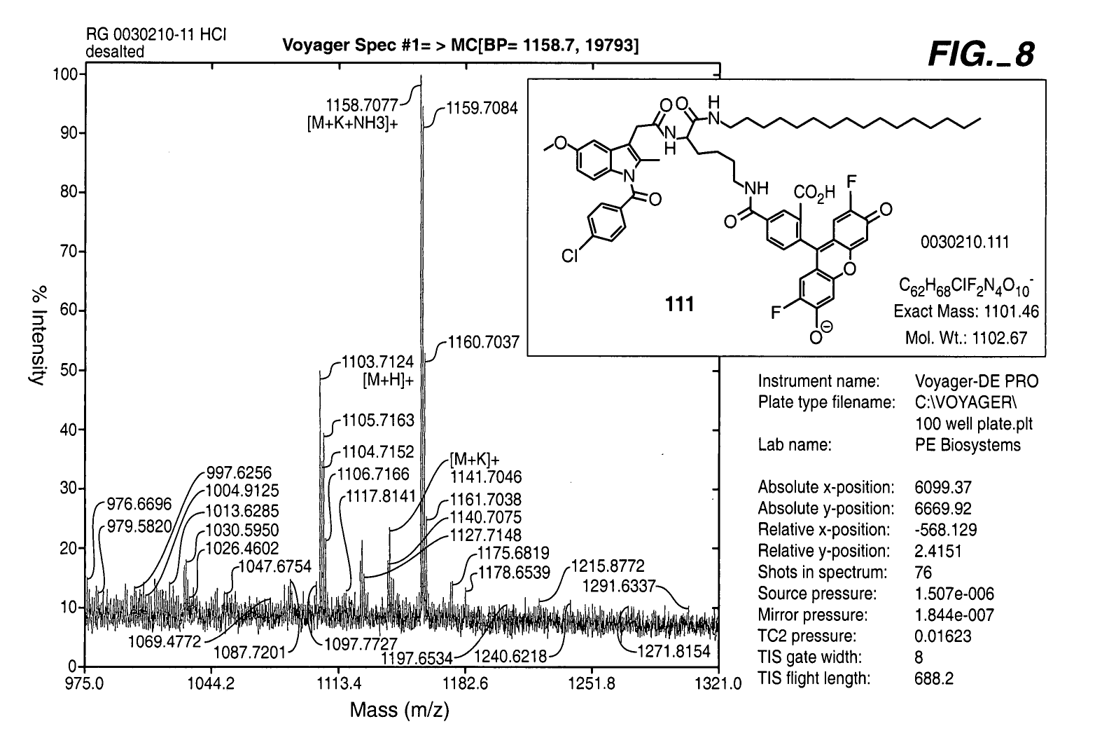

Synthesis of Compound 111. Compound 111 was prepared starting from FMOC-lys(BOC)—OSu in a manner directly analogous to that of compound 107. Indomethacin was purchased from Sigma (part # I 7378). Hexadecylamine was purchased from Aldrich (part # 44,531-2). The synthetic scheme is illustrated in FIG. 5B. The ESI ms was consistent with the assigned structure (see FIG. 8.)

Detection of Binding. Compound 111 (2 ml, 2 nM) in PBS was added to a 3.5 ml quartz cuvette and the fluorescence was measured with a Spex fluorolog 3 fluorimeter. Solutions of compound 111 and increasing amounts of human recombinant COX-2 (Sigma part # C 0858) were prepared and the fluorescence was again measured (FIG. 11). The fluorescence increased about three fold.

### 6.3 Example 3

### Detection of Biotin/Streptavidin Binding Interactions

This example demonstrates the ability to detect binding interactions between biotin and streptavidin using an exemplary binding substrate in which the binding moiety comprises biotin (Compound 103).

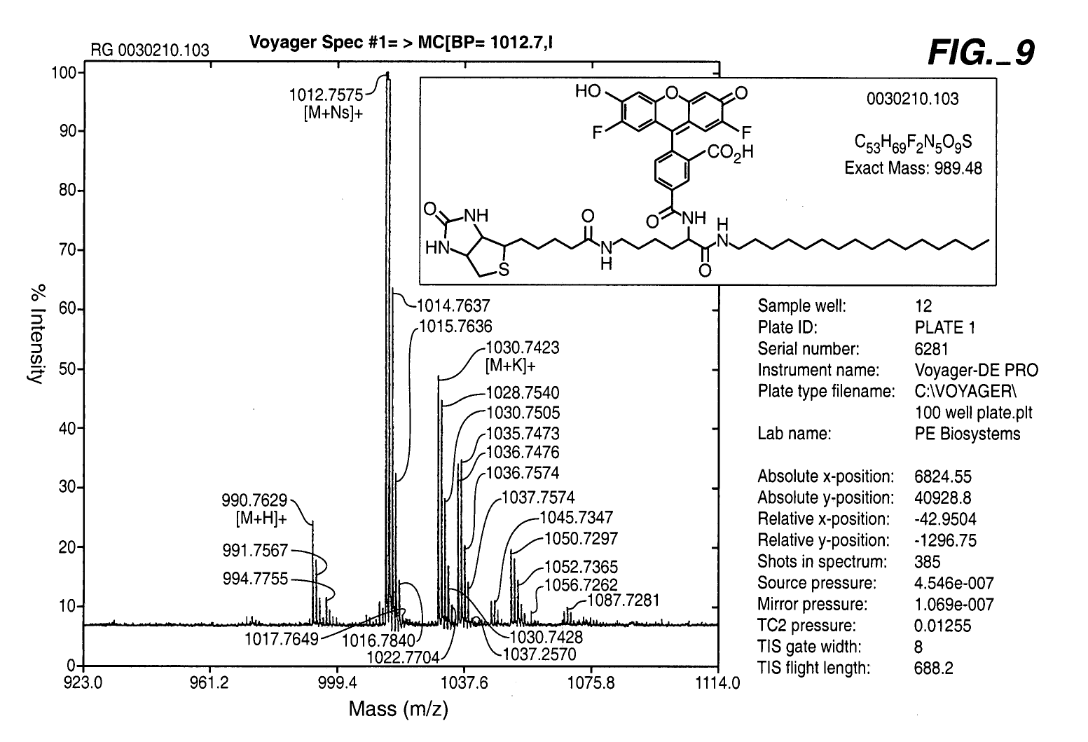

Synthesis of Compound 103. Compound 103 was prepared starting from Fmoc-lys(biotin)-OH (Novabiochem part # 04-12-1237) in a manner analogous to that of Compounds 107 and 111. The synthetic scheme is illustrated in FIG. 5A. The ESI ms was consistent with the assigned structure (see FIG. 9)

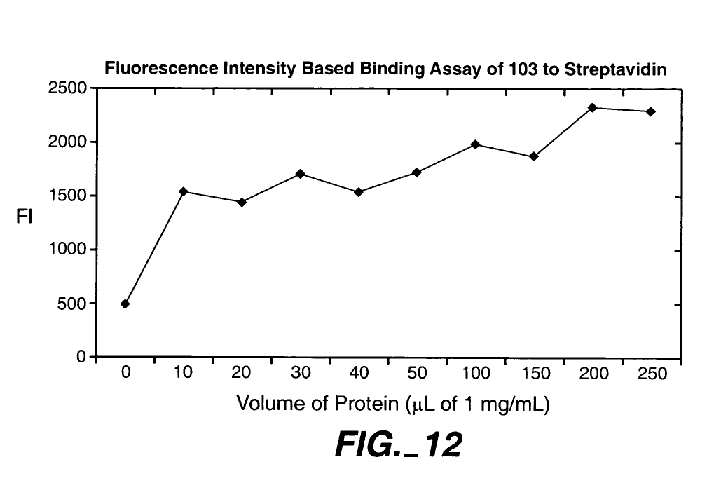

Detection of Binding. Compound 103 (2 ml, 2 nM) in PBS was added to a 3.5 ml quartz cuvette and the fluorescence was measured with a Spex fluorolog 3 fluorimeter. Solutions of compound 103 and increasing amounts of streptavidin (Molecular Probes part # S-888) were prepared and the fluorescence was again measured (FIG. 12). The fluorescence increased about three fold. A similar experiment performed with streptavidin purchased from Pierce Chemicals (catalog no. 21122) did not yield a measurable increase in fluorescence.

### 6.4 Example 4

### Continuation of Micelle Formation

This example demonstrates that, in the absence of a binding partner, the binding substrates described herein form micelles in aqueous buffer, resulting in quenching of their fluorescence signals.

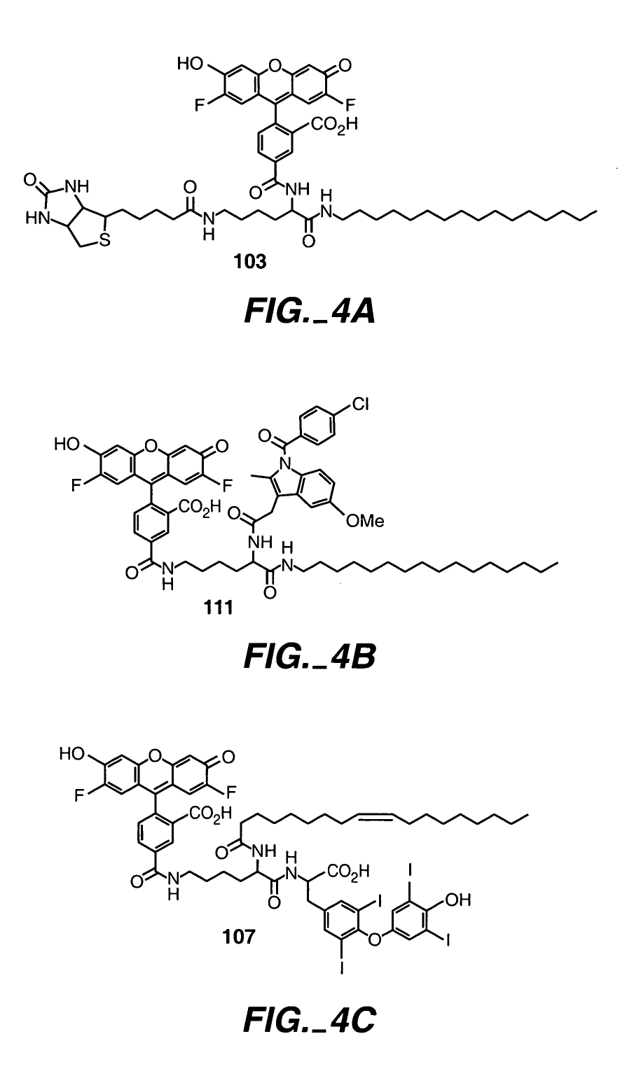

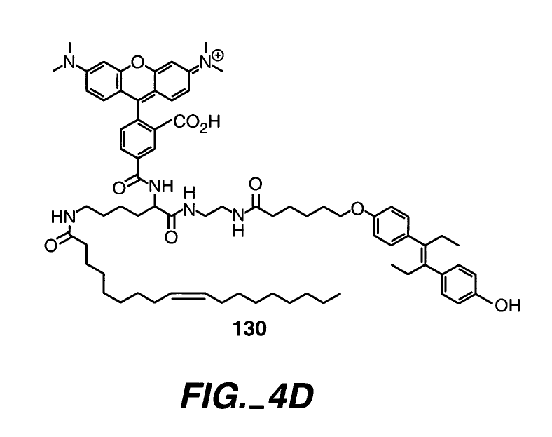

Synthesis of Compound 130. Compound 130 was prepared starting from Fmoc-lys(Boc)—OSu in a manner directly analogous to that of compounds 107 and 111. Diethylstilbestrol was purchased from Sigma (part # D 4628). The structure of Compound 130 is provided in FIG. 4D. The synthesis of Compound 130 is illustrated in FIG. 5D.

Detection of micelles. Compound 130 (1 mg, 8×10⁻⁴ mmol) was dissolved in methanol (1 ml). The stock solution of 130 was serial diluted with PBS to give a final concentration of 2 μM. The absorbance spectra was measured using a quartz cuvette (3.5 ml) and a Cary 3E uv-vis spectrophotometer. Triton X-100 (10 μl, 10% aqueous) was added and the absorbance spectra was measured again. The spectra taken in PBS showed an elevated baseline indicative of light scattering caused by micelles. The spectra taken in PBS/triton X showed a lower baseline indicating that the micelles were dispersed. Triton X-100 is a detergent that breaks up micelles.

The PBS solution of 130 was diluted another two thousand fold (1 nM) and the fluorescence spectra was measured on a Spex fluorolog 3 in a quartz cuvette (3.5 ml). Triton X-100 (10 μl, 10% aqueous) was added and the fluorescence spectra was measured again. Addition of triton X-100 resulted in a 100 fold increase in the fluorescence intensity which indicates that the fluorescence quenching was due to micelle induced quenching.

The experiment was repeated with compound 111. Similar results were found for both 111 and 130.

1. A micelle comprising at least one binding substrate that comprises a hydrophobic moiety capable of integrating the binding substrate into the micelle, a fluorescent moiety and a binding moiety, wherein the fluorescence signal of the micelle is quenched in the absence of a binding partner for the binding moiety of the binding substrate.

2. The micelle of claim 1 in which the hydrophobic moiety comprises a substituted or unsubstituted, saturated or unsaturated hydrocarbon containing from 6 to 30 carbon atoms.

3. The micelle of claim 2 in which the hydrocarbon is a linear, branched or cyclic, saturated or unsaturated alkyl.

4. The micelle of claim 3 in which the alkyl is linear and contains from 10 to 26 carbon atoms.

5. The micelle of claim 4 in which the alkyl is a fully saturated n-alkyl.

6. The micelle of claim 4 in which the alkyl includes one or more carbon-carbon double bonds, each of which may, independently of the others, be in the cis or trans configuration and/or one or more carbon-carbon triple bonds.

7. The micelle of claim 1 in which the hydrophobic moiety comprises a phospholipid.

8. The micelle of claim 7 in which the phospholipid is a sphingolipid.

9. The micelle of claim 7 in which the phospholipid is a glycerophospholipid.

10. The micelle of claim 1 in which the hydrophobic moiety and the binding moiety are linked to one another through the fluorescent moiety.

11. The micelle of claim 1 in which the hydrophobic moiety and the fluorescent moiety are linked to one another through the binding moiety.

12. The micelle of claim 1 in which the hydrophobic moiety, the fluorescent moiety and the binding moiety are linked to one another via a trivalent linker.

13. The micelle of claim 12 in which the trivalent linker comprises an amino acid.

14. The micelle of claim 12 in which the trivalent linker is provided by a trivalent linker synthon illustrated in FIG. 1 G.

15. The micelle of claim 12 in which the binding substrate is selected from a binding substrate depicted in any one of FIGS. 1A-1F, wherein “B” comprises the binding moiety, “D” comprises the fluorescent moiety and R¹ comprises the hydrophobic moiety.

16. The micelle of claim 12 in which the hydrophobic moiety comprises a substituted or unsubstituted, saturated or unsaturated hydrocarbon containing from 6 to 30 carbon atoms.

17. The micelle of claim 16 in which the hydrocarbon is a linear, branched or cyclic, saturated or unsaturated alkyl.

18. The micelle of claim 17 in which the alkyl is linear and contains from 10 to 26 carbon atoms.

19. The micelle of claim 18 in which the alkyl is a fully saturated n-alkyl.

20. The micelle of claim 17 in which the alkyl includes one or more carbon-carbon double bonds, each of which may, independently of the others, be in the cis or trans configuration and/or one or more carbon-carbon triple bonds.

21. The micelle of claim 12 in which the hydrophobic moiety comprises a phospholipid.

22. The micelle of claim 21 in which the phospholipid is a sphingolipid.

23. The micelle of claim 21 in which the phospholipid is a glycerophospholipid.

24. The micelle of claim 1 in which the binding moiety comprises one member of a receptor-ligand pair, or a binding fragment thereof.

25. The micelle of claim 24 in which the binding moiety comprises the ligand.

26. The micelle of claim 24 in which the binding moiety comprises the receptor, or a binding fragment thereof.

27. The micelle of claim 1 in which the binding moiety comprises a candidate compound whose ability to bind another molecule is sought to be determined.

28. The micelle of claim 1 in which the fluorescent moiety comprises a dye having net hydrophilic character.

29. The micelle of claim 1 in which the fluorescent moiety comprises a dye selected from a xanthene dye, a rhodamine dye, a fluorescein dye, a cycanine dye, a phthalocyanine dye, a squaraine dye and a bodipy dye.

30. The micelle of claim 1 in which the fluorescent moiety comprises a xanthene dye.

31. The micelle of claim 30 in which the xanthene dye is a fluorescein dye.

32. The micelle of claim 30 in which the xanthene dye is a rhodamine dye.

33. The micelle of claim 1 in which the fluorescent moiety comprises a fluorescence donor moiety and a fluorescence acceptor moiety.

34. The micelle of claim 33 in which the fluorescence donor moiety comprises a fluorescein dye.

35. The micelle of claim 33 in which the fluorescence acceptor moiety comprises a fluorescein or a rhodamine dye.

36. The micelle of claim 35 in which the fluorescence donor moiety comprises a fluorescein dye.

37. The micelle of claim 1 in which the fluorescent moiety comprises fewer than 150 atoms.

38. A method of detecting the presence and/or quantity of a binding compound in a sample, comprising the steps of:
contacting the sample with a composition comprising a binding substrate that comprises a hydrophobic moiety capable of integrating the binding substrate into a micelle, a binding moiety and a fluorescent moiety, under conditions effective to permit binding between the binding moiety and a binding molecule therefore, if present in the sample; and
detecting a fluorescence signal, where a change in the fluorescence signal indicates the presence and/or quantity of a binding compound in the sample.

39-64. (canceled)

65. A method of identifying and/or characterizing a modulator of a binding interaction, comprising the steps of:
contacting a sample comprising a candidate compound with a composition comprising: (i) a binding substrate that comprises a hydrophobic moiety capable of integrating the binding substrate into a micelle, a fluorescent moiety and a binding moiety; and (ii) a binding partner for the binding moiety, under conditions effective to permit binding between the binding moiety and the binding partner; and
detecting a fluorescence signal, where an increase or a decrease in the fluorescence signal as compared to a control reaction or a standard curve indicates that the candidate modulator compound modulates the binding interaction between the binding moiety and the binding partner.

66-106. (canceled)

107. A method of identifying a compound that binds a receptor of interest, comprising the steps of:
contacting a sample comprising the receptor with a composition comprising a plurality of micelles, each of which comprises a binding substrate that comprises a hydrophobic moiety capable of integrating the binding substrate into a micelle, a fluorescent moiety and a binding moiety comprising a candidate binding compound, wherein the fluorescence spectra of the fluorescent moieties on the micelles are resolvable from one another and are correlated with the structure of the candidate compound comprising their binding moieties, under conditions effective to permit binding between the micelles and the receptor;
detecting a fluorescence signal, where an increase in the fluorescence signal indicates that a micelle comprises a binding compound for the receptor; and
determining the structure and/or identity of the binding compound based upon the detected fluorescence spectrum.

108-139. (canceled)
